(19) United States
(12) Patent Application Publication
(10) Pub. No.: US 2017/0360578 A1
(43) Pub. Date: Dec. 21, 2017

(54) SYSTEM AND METHOD FOR PRODUCING CLINICAL MODELS AND PROSTHESES

(71) Applicant: James Shin, New York, NY (US)

(72) Inventors: James Shin, New York, NY (US); Eugene Jang, New York, NY (US)

(21) Appl. No.: 15/533,092

(22) PCT Filed: Dec. 3, 2015
(86) PCT No.: PCT/US15/63637

§ 371 Date: Jun. 5, 2017

Related U.S. Application Data
(60) Provisional application No. 62/087,638, filed on Dec. 4, 2014.

(51) Int. Cl. A61F 2/50; B33Y 50/00; B33Y 80/00; B29C 64/386; G09B 23/28; B33Y 10/00; B33Y 30/00; G09B 23/30; B29L 31/00; A61F 2/58; A61F 2/66

(52) U.S. Cl. CPC A61F 2/5046; B33Y 30/00; B33Y 50/00; B33Y 80/00; B29C 64/386; G09B 23/30; G09B 23/286; B33Y 10/00; A61F 2/583; A61F 2002/5049; A61F 2002/505; A61F 2002/6614; B29L 2031/7532; A61F 2/66

(57) ABSTRACT

An example method for producing a prosthetic device for a patient includes obtaining imaging data corresponding to a body part of a patient, generating an object model corresponding to the body part based on the imaging data, generating a prosthesis model based on the object model, generating a set of instructions based on the prosthesis model, and executing the set of instructions using a three-dimensional printer, where the set of instructions, when executed by the three-dimensional printer, cause the three-dimensional printer to produce the prosthetic device for the patient.

### TECHNICAL FIELD

This disclosure relates to medical devices, and more particularly to producing clinical models and prostheses.

### BACKGROUND

A clinical model is a representation of a patient's anatomy, pathology, and/or function. For example, a clinical model can represent the structure of one or more tissues, organs, systems, body part, and/or other portions of a patient's body. Clinical models are often used to visualize and simulate the patient's physical and/or functional characteristics, such that the patient can be treated more effectively. For example, a physician may use a clinical model of a patient's heart to better understand the patient's cardiac function, and use that information to render a diagnosis. As another example, a physician may use a clinical model of a patient's knee to diagnose a structural problem with the knee, and use that information to plan a surgical intervention. Clinical models can be physical objects and/or virtual constructs. For example, a clinical model can be a physical model that mimics the three-dimensional structure of a patient. As another example, a clinical model can be a computerized model that mathematically depicts the anatomy, pathology, and/or function of a patient in an electronic medium.

In some cases, a clinical model can be used to produce a prosthesis. A prosthesis is an artificial device that replaces or augments a missing or impaired part of the body. This can occur in a variety of settings including trauma, disease, or congenital conditions. In many circumstances, a prosthesis can be used to partially or fully restore lost functionality, stabilize or support an injured body part to allow more effective healing, or guide the functional or developmental rehabilitation of a body part over time (e.g., in order to regain function following a stroke, or correct a congenital deformity of a limb or posture).

An effective prosthesis should be designed according to the patient's anatomy and functional needs. However, custom manufacturing techniques and personalized fitting procedures can be expensive and often require substantial infrastructure (e.g., a network of accessible clinicians, a prosthesis supply chain, rehabilitation specialists, electricity). In some cases, this can be problematic. For example, in developing regions, a custom prosthesis might be prohibitively difficult to obtain.

### SUMMARY

In general, in an aspect, a method for producing a prosthetic device for a patient includes obtaining imaging data corresponding to a body part of a patient, generating an object model corresponding to the body part based on the imaging data, generating a prosthesis model based on the object model, generating a set of instructions based on the prosthesis model, and executing the set of instructions using a three-dimensional printer, where the set of instructions, when executed by the three-dimensional printer, cause the three-dimensional printer to produce the prosthetic device for the patient.

In general, in another aspect, a system for producing a prosthetic device for a patient includes one or more data processing apparatuses. The data processing apparatus are configured to obtain imaging data corresponding to a body part of a patient, generate an object model corresponding to the body part based on the imaging data, generate a prosthesis model based on the object model, generate a set of instructions based on the prosthesis model, and execute the set of instructions using a three-dimensional printer, where the set of instructions, when executed by the three-dimensional printer, cause the three-dimensional printer to produce the prosthetic device for the patient.

Implementations of these aspects may include or more of the following features.

In some implementations, the method can further include identifying supplemental data based on the received imaging data and/or the object model, where supplemental data comprises supplemental imaging data corresponding to body parts of one or more other patients, and updating the object model based on the imaging data and the supplemental data. In some implementations, the processor can be further configured to perform these steps.

In some implementations, the method can further include identifying supplemental data based on the received imaging data, where supplemental data comprises supplemental imaging data corresponding to body parts of one or more other patients, and where generating an object model is further based on supplemental data. In some implementations, the processor can be further configured to perform these steps.

In some implementations, the imaging data and the supplemental imaging data can be each acquired using the same imaging modality. In some implementations, the imaging data and the supplemental imaging data can be each acquired using a different imaging modality. In some implementations, the imaging modality can be photography, computed tomography, magnetic resonance imaging, ultrasound, and/or X-ray.

In some implementations, the imaging data and the supplemental imaging data can correspond to similar body parts.

In some implementations, the object model can include surface information.

In some implementations, the object model can include mesh information.

In some implementations, the object model can include information regarding a plurality of components. The object model can include information regarding a dynamic interaction between one or more of the components.

In some implementations, generating the object model can include determining the object model using photogrammetry.

In some implementations, generating the object model can include segmenting the imaging data into one or more portions. The one or more portions can each correspond to a different bone or soft tissue structure in the body party.

In some implementations, generating the object model can include modeling a surface of the body part.

In some implementations, generating the object model can include estimating a volume of the body part.

In some implementations, identifying supplemental data can include determining a similarity between the supplemental imaging data and the imaging data. The supplemental imaging data and the imaging data can correspond to body parts having similar physical characteristics. The similar physical characteristics can include a similar spatial dimension. The similar physical characteristics can include a similar volume. The similar physical characteristics can include a similar shape.

In some implementations identifying supplemental data can include determining a similarity between demographic data corresponding the patient and demographic data corresponding to one or more other patients.

In some implementations, the set of instructions, when executed by the three-dimensional printer, can cause the three-dimensional printer to produce the prosthetic device for the patient through an additive manufacturing process.

In some implementations, generating the object model can include transmitting the imaging data to a remote processing device, and receiving the object model from the remote processing device, where the object model is generated by the remote processing device based on the imaging data.

In some implementations, generating a prosthesis model based on the object model can include transmitting the object model to a remote processing device, and receiving the prosthesis model from the remote processing device, where the prosthesis model is generated by the remote processing device based on the object model.

In some implementations, generating the set of instructions based on the object model can include transmitting the prosthesis model to a remote processing device, and receiving the set of instructions from the remote processing device, where the set of instructions is generated by the remote processing device based on the prosthesis model.

In some implementations, generating the object model, generating the prosthesis model based on the object model, and generating the set of instructions based on the prosthesis model can include generating the object model, the prosthesis model, and the set of instructions through the use of a remote processing device.

In general, in another aspect, a method for producing an object model of a patient includes obtaining a first set of imaging data corresponding to a body part of a patient. The first set of imaging data was acquired using a first imaging modality. The method also includes obtaining a second set of imaging data corresponding to the body part of the patient. The second set of imaging data was acquired using a second imaging modality different than the first imaging modality. The method also includes generating an object model corresponding to the body part based on the first set of imaging data and the second set of imaging data.

Implementations of these aspects may include or more of the following features.

In some implementations, the object model can be generated by identifying portions of the body part from the first set of imaging data and identifying different portions of the body part from the second set of imaging data and generating a composite set of data based on the identifications. The portions of the body part from the first set of imaging data can correspond to different tissue types than the portions of the second body part. The tissue types can be selected from the group consisting of bone, connective tissue, vascular tissue, muscle tissue, nerve tissue, and epithelial tissue. The portions of the body part can be manually identified. The portions of the body part can be identified from the first set of imaging data have higher image contrast using the first imaging modality than the second imaging modality. The portions of the body part can be identified from the second set of imaging data have higher image contrast using the second imaging modality than the first imaging modality.

In some implementations, the object model can include information regarding a surface of the body part.

In some implementations, the object model can include information regarding a volume of the body part.

In some implementations, the object model can include information regarding a spatial dimension of the body part.

In some implementations, the object model can include information regarding a geometric shape of the body part.

In some implementations, generating the object model can include registering the first set of imaging data and the second set of imaging data to a common geometric space. The first and second sets of imaging data can be registered based on a location of one or more common body part features in the first and second sets of imaging data. Generating the object model can further include identifying one or more first anatomical structures based on the first set of imaging data, identifying one or more second anatomical structures based on the second set of imaging data, and generating the object model based on the identified first anatomical structures and second anatomical structures. Identifying one or more first anatomical structures based on the first set of imaging data can include segmenting the first set of imaging data based on one or more properties of the first set of imaging data. The one or more properties can include at least one of: a localized image intensity, a localized image contrast, and a localized geometric shape.

In some implementations, the method can further include generating a set of instructions for a three-dimensional printer based on the object model, and executing the set of instructions using a three-dimensional printer. The set of instructions, when executed by the three-dimensional printer, cause the three-dimensional printer to produce a physical model of the body part.

In some implementations, either the first imaging modality or the second imaging modality can be photography.

In some implementations, either the first imaging modality or the second imaging modality can be computed tomography.

In some implementations, either the first imaging modality or the second imaging modality can be magnetic resonance imaging.

In some implementations, either the first imaging modality or the second imaging modality can be X-ray.

In some implementations, either the first imaging modality or the second imaging modality can be ultrasound.

Among other advantages, some implementations may allow users to design and produce customized models and prostheses quickly, cost effectively, and/or with a reduced reliance on infrastructure. As such, customized models or prostheses can be produced even in regions with limited infrastructure or resources. These qualities are beneficial, for example, in developing regions where monetary, infrastructure, and health care access challenges may otherwise preclude patients from obtaining a suitable prosthesis. These qualities are also beneficial in developed countries; for example, clinical models or prostheses can be generated at a reduced cost, such that clinical models or prostheses are more readily available while lowering the overall cost of treatment. Further, in some implementations, a customized model or prosthesis can be designed for a patient based on imaging data obtained from multiple other patients having similar characteristics. In this manner, customized models or prostheses can be produced for patients that might otherwise have limited access to imaging equipment.

The details of one or more implementations are set forth in the accompanying drawings and the description below. Other features and advantages will be apparent from the description and drawings, and from the claims.

### DESCRIPTION OF DRAWINGS

FIGS. 1-3 are diagrams of example processes for fabricating a prosthesis.

FIG. 4A shows an example photograph of a patient's body part.

FIG. 4B shows an example representation of a point cloud generated based on several photographs of a patient's body part.

FIG. 4C shows an example representation of an object model of a patient's body part.

FIG. 4D shows an example prosthetic device.

FIG. 4E shows another example prosthetic device.

FIG. 5 is a diagram of an example process for producing an object model of a patient using multiple different imaging modalities FIGS. 6A-C are diagrams of an example systems for fabricating a prosthesis.

FIG. 7 is a diagram of an example computer system.

### DETAILED DESCRIPTION

Implementations for fabricating patient-specific clinical models and prostheses are described below. The term “patients” refers to both human and non-human patients (e.g., pets, such as dogs, horses, cats, etc.). One or more implementations can use automated and/or semi-automated and computer aided design (CAD) processes to enable rapid turnaround between the design and production of a model or prosthesis.

In some implementations, clinical models or prostheses can be produced based on information obtained using multiple different imaging modalities. This can be beneficial, for example, as a patient's anatomy and pathology are often highly complex. As different imaging modalities may each provide potentially unique information regarding the anatomy and pathology of the patient, the individual strengths of each imaging modality can be leveraged to produce a more accurate model or prosthesis.

In some cases, implementations can be used to provide physicians or patients with customized models or prostheses at a reduced cost and/or with a reduced reliance on infrastructure. As an example, a cost-effective and easily deployed customization and fabrication technique can be used to provide individualized model or prosthesis fabrication capabilities, even in regions with limited infrastructure or resources. These qualities are beneficial, for example, in developing regions where monetary, infrastructure, and health care access challenges may otherwise preclude patients from obtaining a suitable prosthesis, or preclude physicians from obtaining accurate clinical models. These qualities are also beneficial in developed countries; for example, clinical models or prostheses can be generated at a reduced cost, such that clinical models or prostheses are more readily available while lowering the overall cost of treatment. Further, in some implementations, a customized model or prosthesis can be designed for a patient based on imaging data obtained from multiple other patients having similar characteristics. In this manner, customized models or prostheses can be produced for physicians or patients that might otherwise have limited access to imaging equipment.

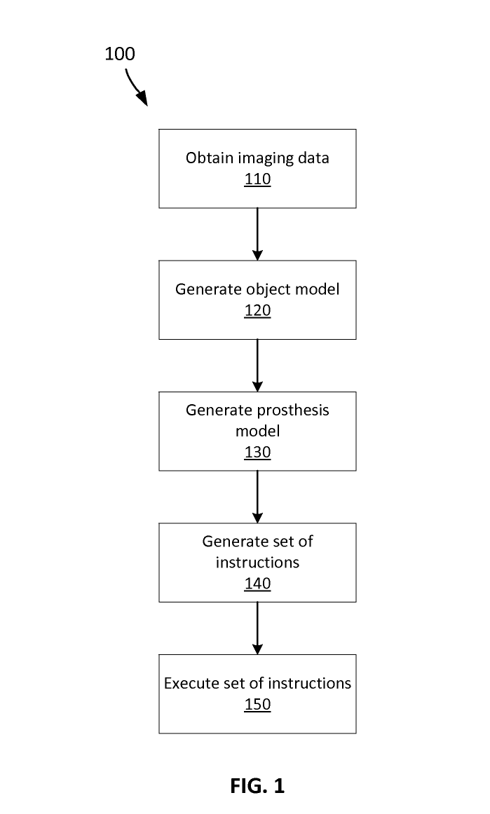

An example process 100 for fabricating a prosthesis is shown in FIG. 1. The process 100 begins by obtaining imaging data corresponding to a body part of a patient (step 110). One or more different types of imaging data can be obtained, including photographs (PX), computed tomography (CT) data, magnetic resonance imaging (MRI) data, ultrasound (US) data, and X-ray radiographs (XR), among others.

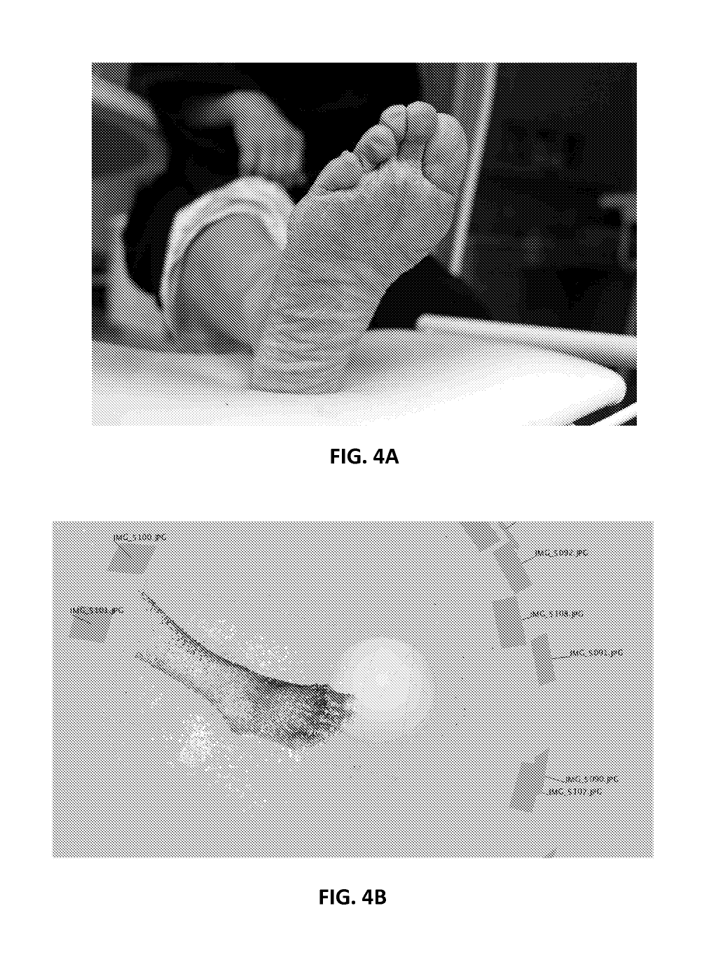

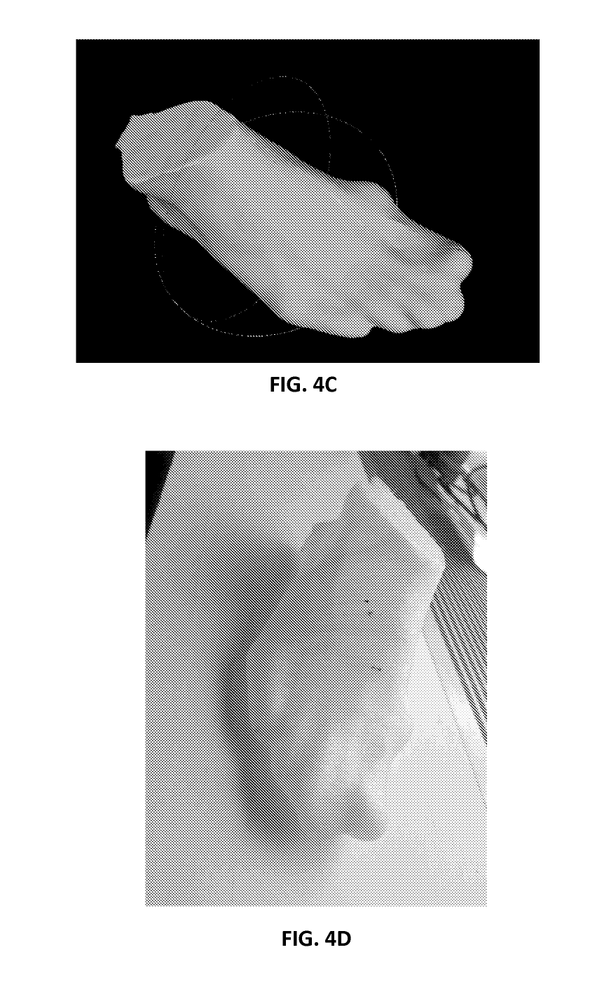

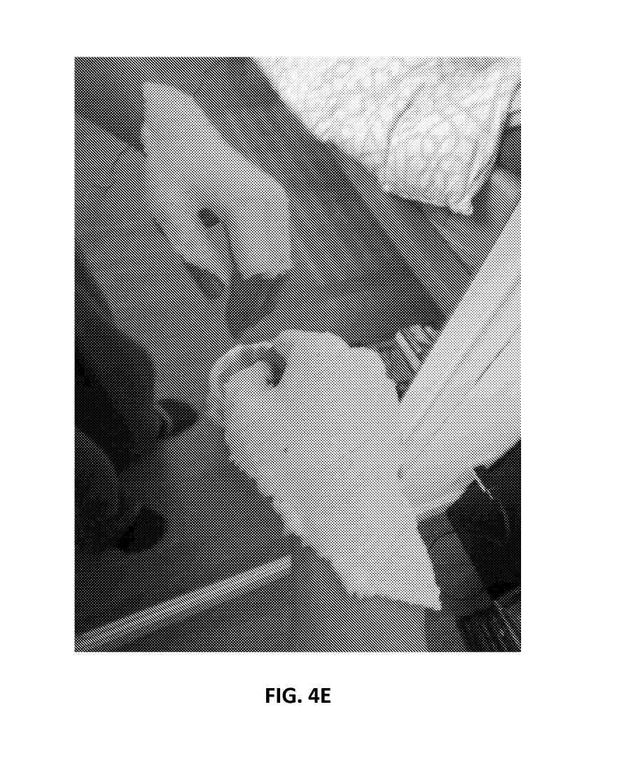

For example, in some implementations, photography (such as film or digital photography) can be used to capture photographs of a body part of the patient. Photographs can be captured from a variety of different vantage points, such that the photographs, as a whole, capture visual information about the body part from multiple perspectives. Photographs can be captured evenly about the body part; for example, each photograph's perspective can be separated from another photograph's perspective by a fixed angular distance. Photographs can also be captured irregularly about the body part; for example, each photograph's perspective can be separated from another photograph's perspective by an arbitrary angular distance. Likewise, each photograph can be captured from a fixed distance away from the body part, or each photograph can be taken from varying distances away from the body part. In an example implementation, contiguous overlapping images of a body part are taken from roughly 10-15 degrees off-axis in a circular and/or spherical plane, until the body part has been imaged from every relevant side. Although in some implementations, only a certain number of photographs might be needed to complete the process, additional photographs can also be captured to provide oversampling of relevant detail and to provide backup photographs in case certain photographs are later found to be unsuitable. In some implementations, photographs can be captured using multiple fixed cameras, cameras on a revolving axis, or other means of controlling the position of the camera relative to the body part of interest, and relative to subsequent camera angles. As an example, FIG. 4A shows a photograph of a patient's foot. As described above, several similar photographs, each depicting the patient's foot from a different vantage point, can be obtained during step 110.

In some implementations, photographs can be captured in a manner that maintains uniform (or otherwise similar) exposure. Likewise, in some implementations, photographs can be captured with appropriate apertures for focus depth and shutter speeds to reduce or eliminate blur.

In some implementations, surface detail of the body part may be difficult to register (e.g., in the case of relatively uniform or smooth surfaces), or various features of the body part can be difficult to discern. In these cases, “landmarks” can be added to the body part in order to provide a visual point of reference. For example, the body part can be marked (e.g., with a pen or stickers), such that the marked area acts as a common point of reference across multiple photographs. Surface markings can also provide a common dimensional scale across multiple photographs. In some cases, body parts that are difficult to register due to hair or fur (e.g., body parts such as the head, or body parts of animals or hirsute individuals), can be prepared using measures to remove all or part of the hair (e.g., shaving), or using hair-preserving techniques that reduce the impact of the hair on registration (e.g., wrapping a tight, thin bandage or other material to approximate surface topography).

Photographs can be captured using a film camera, a digital camera, or a digital image capture component integrated into another electronic device (e.g., an image capture module in a cellular phone, tablet computer, computer, webcam, or other electronic device). In some implementations, photographs can be captured using a video camera (e.g., a digital video camera) that captures multiple frames of imaging data in rapid succession; in these implementations, individual photographs can be extracted by isolating all or some of the image frames.

In another example, imaging data can be obtained using medical imaging modalities such as CT, MRI, ultrasound, and X-ray. In general, two dimensional image data acquired at regular and/or known intervals can be reconstructed to approximate a three dimensional volume. Such volume information, when acquired repeatedly and in sequence, may additionally provide temporal information often referred to as the “fourth dimension” in four dimensional or functional imaging. Cross-sectional imaging techniques such as CT and MRI, for example, can in this way provide high-resolution soft tissue and bone information in two, three, or four dimensions. Ultrasound imaging techniques can similarly provide morphologic information in two, three, or four dimensions. Imaging techniques such as X-ray radiography can also provide soft tissue and bone information, typically in two dimensions. Imaging techniques may also be employed to directly ascertain surface and depth information regarding a body part with remote sensing technologies utilizing various spectra of electromagnetic radiation (e.g., laser scanner, time-of-flight imaging, or other form of “light radar” or LiDAR), accessible as commercial systems (Sense, NextEngine, Fuel3D, IIIDScan), as well as aftermarket open-source modifications of consumer hardware (e.g., OpenKinect modification of Microsoft Kinect).

In some implementations, imaging data from multiple image modalities can be obtained. For example, in some implementations, both photographs and CT data can be obtained for a patient. In another example, photographs, MRI data, and X-ray data can be obtained for a patient.

Further, imaging data need not be obtained contemporaneously. For example, in some implementations, imaging data can be obtained from different points in the past (e.g., photographs can be taken during a present medical examination, while CT data can be taken during a different medical examination at some time in the past).

Further still, imaging data can be obtained by accessing imaging data from a storage facility (e.g., a server computer or repository that stores imaging data for one or more patients). As an example, imaging data can be obtained by transmitting the imaging data from a server computer to a client computer. Imaging data can be transmitted, for example, using a communications network (e.g., a cellular telephone network, a local area network, a wide area network, a WiFi network, a Bluetooth network, or other communications network) or via physical transmission (e.g., by sending imaging data through a postal system, exchanging physical images, or exchanging data storage devices containing imaging data).

The images can be of different formats, depending on the implementation. For example, photographs might be stored as JPG, RAW, TIFF, or BMP files, while X-ray radiographs, ultrasound data, CT data, and MRI data might be stored as DICOM files or as binary data. In some implementations, one or more of the images may be obtained from a Picture archiving and communication system (PACS), which can store images corresponding to several different patients and several different imaging modalities. Other image formats and image storage systems can also be used, depending on the implementation, and may include specialty or proprietary formats requiring additional processing or conversion into another format.

After imaging data is obtained, the process 100 continues by generating an object model corresponding to the body part based on the imaging data (step 120). An object model is a mathematical representation of the body part, and describes in three-dimensions the physical characteristics of the body part. For example, the object model can include information regarding the volume of the body part (e.g., the space enclosed by the body part). In some implementations, the object model can include information regarding the surface of the body part (e.g., the shape of the body part, the outer surface contours of the body part, the texture of the body part, and so forth). In some implementations, the object model can include information regarding multiple components contained within the body part. For example, for a patient's leg, an object model can include information regarding the location and shape of one or more bones, one or more articular surfaces, one or more ligaments, one or more tendons, and so forth. In some implementations, the object model can also include information regarding a dynamic interaction between each of the components. For example, for the object model of the leg, the object model can include information regarding the interaction between each of the bones, ligaments, articular surfaces, and/or tendons as the leg is moved between different positions or subjected to different degrees of external force.

An object model can be generated in a variety of ways. For example, photogrammetry can be used to extract positions of various surface points of the body part in order to produce an object model that represents that body part. In an example implementation, photogrammetry can be used to analyze the parallax between common registered points across multiple photographs taken from different angles, and extract coordinates corresponding to specific points on the body part's surface in three-dimension space. Once a sufficient number of points have been determined to form a “point cloud” (e.g., a collection of points) with sufficient detail, the points can be used to create an object model that includes mesh information, surface contour information, and/or solid body information corresponding to the body part. For example, the points of the point cloud can be interconnected, such that they form a mesh that approximates the body part's surface. In another example, the space enclosed by the point cloud can approximate the volume of the body part. In some implementations, object models can be generated from point clouds using free and/or commercially available CAD software, such as MeshLab, Blender, Solidworks, and PhotoScan. As an example, FIG. 4B shows a representation of a point cloud generated based on several photographs of a patient's foot, and FIG. 4C shows a corresponding object model of the patient's foot.

In some implementations, photogrammetry can utilize landmarks inherent to the body part (e.g., distinct physical features of the body part) and/or landmarks that were added to the body part during image data acquisition (e.g., markers that were added to the body part using a pen or sticker prior to photographing) in order to register or align multiple photographs with one another. Likewise, landmarks can be used to ascertain a reference scale between multiple photographs in order to account for features that may appear larger or smaller between different photographs due to varying distances from the image capture device.

In some implementations, manual, semi-automated, or fully automated pre-processing of images can be used to create an object model. For example, in some implementations, a user can mask the imaging data such that extraneous or background detail is removed. In another example, a semi-automated process can be used to predict which portions of a photograph correspond to extraneous information, and the user can manually select which portions to remove. In yet another example, a fully automated process can be used to predict which portions of the photograph correspond to extraneous information and remove those portions automatically. In some cases, masking the imaging data can increase the accuracy and speed of the object modeling process.

In some implementations, an initial low-pass analysis of distinct image detail can be performed. This data can be compared among images in the image set in order to identify relative camera position, for example, by comparing the parallax of adjacent points acquired at different angles. In some cases, no a priori assumptions about relative camera positions or foreground/background detail need be made, and an arbitrary sparse point cloud of the structure can be generated based solely on the imaging data. However, should camera positions be known or fixed, such as when using a single or multi-camera stage, this information can assist in position registration. In certain instances, this additional position information can increase the speed of processing and accuracy of point registration. In some implementations, each calculated or known camera position and its registered points can be represented in three-dimensions, such that extraneous or mis-registered points can be easily removed from the point cloud, either as clustered points (e.g., by selectively disregarding portions of the point cloud itself), or as groups of points registered from a single angle of acquisition (e.g., in the instance of an individual image that is of poor quality or is otherwise suboptimal).

In some implementations, a region of interest can additionally be tightly fit to the margins of the body part to be modeled, further excluding extraneous or erroneous points. A dense point cloud can then be generated efficiently, for example, through deeper iterative re-processing by similar calculations registering point detail across images, and the relative parallax of these points at various angles of acquisition. For example, an initial arbitrary maximum point number can be set to a relatively low value, optimizing speed and producing a sparse point cloud that can provide general contours of the object to be modeled. The maximum point number can then be increased in order to obtain the desired smoothness and fidelity with respect to the morphology of the object and/or within the confines of computational hardware capabilities. This process can be repeated in a step-wise fashion, with manual cropping and exclusion of extraneous points that are mis-registered or correctly registered but outside the relevant area of interest. Clusters of points registered from a single image at a particular angle may also be revealed to be suboptimal (e.g., due to object motion at the time of image capture, computational error, subtle differences in exposure, or otherwise), and can be excluded by removing the camera angle and attributed points en-bloc.

In some implementations, specific information regarding the camera's characteristics (e.g., specific imaging characteristics of the camera, specific settings used by the camera to capture the image, or specific characteristics of the camera lens), image characteristics (e.g., known locations of foreground or background information), and object morphology can also improve accuracy. For example, variable depth filtering, lens distortion, and expected degree of parallax can be adjusted to further increase the fidelity of the object model.

In some cases, the object model can be “repaired” or otherwise modified using CAD software. For example, this may entail removing self-intersection faces, decimating the model to a manageable complexity, applying smoothing or other filters, global surface re-meshing, or solidifying or hollowing the model. This may also entail brush-based manipulation, such as spot correction of image or segmentation artifacts, local re-meshing, volume cropping, or vertex painting. This may also entail integration with another object model, for example an object model representing a different area of the same imaging dataset, or an object model made from supplemental data.

The number of points needed to make an object model can differ, depending on the implementation. For example, a large number of points can result in a more detailed and/or accurate object model. However, in some cases, it may be computationally or time prohibitive to determine a large number of points. For each specific implementation, the exact number of points can be varied in order to accommodate these factors.

As noted above, in some implementations, photographs can be captured using multiple fixed cameras, cameras on a revolving axis, or other means of controlling the position of the camera relative to the body part of interest, and relative to subsequent camera angles. Information regarding the known position of each of the cameras can be used to further improve the speed and accuracy of photogrammetry.

In some implementations, radiographic images (e.g., X-ray radiographs) can provide additional soft tissue and bone contour information in two dimensions and illustrate pathology. The addition of radiographic data can further inform design by illustrating areas of bone injury or protuberance, points of probable instability, and optimal points of fixation and stabilization. In some implementations, CT data and MRI data can similarly provide soft tissue and bone morphologic and pathologic information in three-dimensions, and lends itself naturally to object modeling through segmentation. In some implementations, ultrasound data can similarly be modeled through segmentation, for example by segmenting contiguous two-dimensional images comprising a volumetric three dimension acquisition. In some implementations, surface and depth information may be directly ascertained as a surface mesh with various remote sensing technologies such as time-of-flight imaging or laser scanning, forgoing in part or in total the need for individual image segmentation. Together, these modalities (photography, X-ray, CT, MRI, ultrasound, LiDAR) can provide complementary and overlapping information enabling highly customized and dimensionally accurate object models for individual anatomy and/or pathology.

As described above, multimodal imaging data can be segmented in order to provide additional information regarding the body part. In image segmentation, an image is divided into portions in order to define image regions that pertain to particular features of the body part. For example, an image can be segmented such that a first bone is in one segmented region, another bone is in another segmented region, one type of soft tissue is in another segmented region, and so forth. By segmenting an imaging data set, information regarding the location, shape, and interaction between one or more components of the body part can be extracted. This information can then be used to generate more accurate object models of the body part. For example, in some implementations, segmented imaging data can be analyzed using finite element analysis and tolerance analysis in order to predict points of instability or weakness during force bearing and/or movement. Knowledge of tissue mechanical properties including and/or analogous to density, elastic modulus, shear modulus, Poisson's ratio, and material damping ratio, can be assigned to each segmented component based on known, estimated, or measured values. Virtual assembly of each segmented tissue into a limb can thus provide dynamic modeling capabilities through finite element analysis in commercially available CAD software (e.g., Solidworks, 3-maticSTL), facilitating mathematical solutions to complex structural analysis and elasticity problems in an automated and visual fashion. For example, forces corresponding to load bearing, range of motion about joint axes, and morphologic deformations relating to muscle contraction can be virtually applied in a simulated environment, revealing areas of increased stress, areas susceptible to distraction and/or misalignment, and points of potential soft tissue impingement. In some instances, a dynamic object model of a limb such as described can serve as a platform upon which a suitable prosthesis model can be designed. Knowledge of material properties relating to the prosthesis, for example density, elastic modulus, shear modulus, thermal expansion coefficient, yield strength, tensile strength, and Poisson's ratio, can be assigned to the prosthesis and/or its component parts. The prosthesis model can be virtually subjected to analogous forces and motions applied to the object model of the body part, and these forces can be safely explored to the point of mechanical failure in a simulated environment. In some instances, the prosthesis model can be virtually applied to the limb object model, and the dynamic interaction between them can also be examined in a simulated environment. Object modeling in this manner can thus speed the process of design by forgoing costly and time-consuming prototyping, fabrication, and iterative fitting, while maintaining insights derived from an iterative revision process including those related to mechanical robustness, comfort, and safety of the end design. This advantage is of particular benefit in certain applications requiring multiple unique prostheses, for example in the case of pediatric and adolescent patients requiring long-term or serial corrective prostheses, during which time morphology is expected to change with growth and maturation. This is described in greater detail below.

As an illustrative example, CT data is obtained for a patient suffering from a complex ankle fracture. The CT data provides morphologic information regarding the ankle, including bone fragments and their position. The CT data is segmented into different regions, for example to define image regions corresponding to bone, soft tissue, and the background. This segmentation can be performed manually by a user (e.g., by inputting boundaries for each of the image segments), semi-automatically (e.g., by a computer identifying image segments based partially on user direction), or automatically (e.g., by a computer identifying image segments without user direction). Knowledge of the image acquisition parameters (e.g., the resolution of the imaging data, the field of view of the imaging data, the imaging plane, and so forth), knowledge of morphology (e.g., an expected thickness of certain types of tissue, an expected location certain types of tissue), and knowledge pertaining to the imaging modality (e.g., signal intensities of anatomic structures on particular MRI sequences, and linear attenuation coefficients of anatomic structures on CT images) can also be used in order to segment an image. In some implementations, images can be segmented according to threshold values. For example, in CT imaging, bone and soft tissue may be expected to result in different degrees of X-ray beam attenuation; a threshold value can be used to distinguish one type of tissue (e.g., bone) from another (e.g., soft tissue). The efficiency and fidelity of threshold segmentation techniques can be further enhanced through image acquisition techniques including dual-energy CT (simultaneous acquisition of imaging data at different beam energies, as defined by the voltage applied to a beam generator on a CT scanner), wherein characteristic and measurable changes in linear attenuation coefficients of particular tissues at the different beam energies can be used to selectively segment these areas with greater specificity. An initial object model may require further refinement in CAD (e.g., MeshLab, Netfabb, Blender, Rhino3D), for example to remove unwanted artifacts resultant from the process of segmentation. Mesh repair tools including those described above may be applied as necessary until an object model of satisfactory quality is produced. A bone model generated in this manner can be further manipulated within CAD software (e.g., Solidworks, 3-maticSTL), treating segmentally fractured or displaced fragments as independent bodies, which can be repositioned to anatomic or near-anatomic position. Finite element analysis and tolerance analysis of the resulting configuration can help predict points of ankle and fracture instability during load bearing; if imaging data was acquired subsequent to successful reduction (e.g., after restoring a fracture or dislocation to the correct alignment), the existing configuration of bone fragments can be similarly analyzed without repositioning of the bone fragments. In some implementations, further and more complex modeling (e.g., more complex modeling of bone and ligamentous instability resulting from injury to either) can be pursued based on imaging data obtained using additional imaging modalities.

After an object model is generated, the process 100 continues by generating a prosthesis model based on the object model (step 130). In some cases, the prosthesis model can be similar to the object model. For example, in the case of a static prosthesis to replace a patient's missing body part, the prosthesis model can be similar to the object model (e.g., an object model of the missing body part based on imaging data obtained when previously intact, or an appropriately identified surrogate limb) or a mirrored version of the object model (e.g., an object model of the patient's corresponding intact contralateral body part). In some cases, an object model can also provide functional, mechanical, and morphologic information necessary to generate a prosthesis model for a static or functional prosthesis. In some cases, (e.g., for some static prostheses), information regarding limb morphology provides sufficient information to construct a cosmetic prosthesis model, which is appropriately sized with respect to the patient. In some cases, combining this morphologic information of a desired prosthesis model with an object model of an individual's residual body part, in certain cases referred to as a “stump,” may provide sufficient information to design a socket interface between the residual body part and prosthesis. As an example, the object model of the patient's foot shown in FIG. 4C can also be used as a prosthesis model.

In some cases, the prosthesis model can be generated based on the object model, but need not be geometrically or mechanically similar. For example, robust object modeling (e.g., using one or more of techniques described above) may provide a dynamic limb model that serves as a virtual construct around which a supportive prosthetic device (e.g., an orthosis or brace) can be modeled. For example, based on an object model of a limb, a prosthesis model can be generated by considering points of desired reinforcement, relief, aesthetics, and motion. Elements of the prosthesis model can be positioned as appropriate to achieve the desired result.

In some cases, patient comfort may be increased by accurately reproducing the contours of the residual limb in the prosthesis model. In some cases, patient comfort may be increased further through dynamic modeling of the residual limb by methods similar to those described above, in order to reflect deformations to the limb resulting from forces such as load bearing and/or movement. This dynamic modeling may capture changes in limb morphology with movement as well as with swelling, a contributor to discomfort in certain cases. Material properties of segmented soft tissue components can be manipulated to simulate the effects of variable degrees of swelling, analogous to industrial applications of thermal coefficients of expansion for various materials in a multi-material object. Tolerance analysis of these dynamic changes can reveal areas of increased stress at the object-prosthesis interface, further informing design for increased comfort.

Knowledge of the interaction of forces about an anatomic joint that enable functional anatomic movement can inform the design of a functional prosthesis by providing a biomimetic virtual model of anatomic motion. As an example, force generation along muscle belly axes can be represented as linear vectors, individually or as muscle groups, according to the extent of body part preservation and desired complexity of function of the prosthesis. For example, a prehensile hand prosthesis can be designed for a mid-radius amputee, with a single flexion-extension movement simultaneously actuating wrist flexion/grasping and wrist extension/grip opening movement through grouping of force vectors related to wrist, hand, and finger movement. A biomimetic cable pulley system, analogous to the modeled myotendinous architecture of the hand and functioning to reproduce anatomic force vectors, can be anchored to a force-generating mechanism associated with the prosthesis, enabling the desired motion. The individual biomimetic tendons of the fingers and wrist can be combined into flexion/grasping and extension/opening groups, thus simplifying the design of the force-generating component. Increasing complexity of the force-generating component can be designed in such a way that all potential forces about all axes of anatomic motion are represented, thus providing a functionally biomimetic hand prosthesis.

The force-generating component of a prosthetic device may be of various materials and components according to the desired function, strength of force, and manufacturing and usage considerations such as material availability, weight, and cost. As an example, electronic motors integrated into the prosthesis can actuate cables in a cable pulley system analogous to the myotendinous architecture of the modeled limb. As another example, electronic motors integrated into the joints themselves may actuate components of the prosthesis directly, without use of a cable pulley system. As another example, thermocontractile material including coiled polymer fibers such as nylon may include both the cable pulley system and force-generating component together as one. Electroactive polymer fibers such as silver-plated nylon may similarly include both the cable pulley system and force-generating component together as one, and provide a facile method of heating through its conductive and resistive properties. Rapid cooling and thus relaxation may be facilitated by a vented cable pulley system design or through a closed, circulating coolant system surrounding the cables in a cable pulley system. A closed, circulating heating/coolant system could provide similar thermal control in a system utilizing non-electroconductive, thermocontractile coiled polymer fiber pulley system. These systems may be further enhanced through use of highly thermoconductive and electroconductive material such as graphene. The strength of the cable, strength of the force of contraction, and tensile stroke can be designed in a biomimetic fashion according to known biomechanical properties of the anatomic myotendinous architecture, mechanical properties of the cable system, and the desired functionality of the prosthesis. For example, polymer fiber of appropriate gauge and degree of coiling can be used to provide adequate cable strength, force of contraction, and tensile stroke, approximating anatomic function.

In some cases, the force-generating component of a prosthetic device can be coupled to the user in a manner in which movement can be volitional, for example mechanical coupling of the functionality of the prosthesis to the movement of an existing intact joint. For example, a hand prosthesis designed for a mid-radius amputee can be designed with an extension of the socket to the upper arm, in such a way that flexion and extension at the intact elbow initiates wrist extension/grip opening, and flexion at the intact elbow initiates wrist flexion/grasping. This mechanical coupling may trigger a force-generating component within the prosthesis, or could itself be the force-generating component. A cable pulley system could be designed in such a way that the amplitude of motion at the intact joint is appropriately amplified or dampened in accordance with the desired range of motion and/or force of the functional prosthesis.

In some cases, a microcontroller integrated into the prosthesis can translate mechanical and/or myoelectric coupling of the residual limb to the functional prosthesis. For example, electrodes sensitive to signals generated within nerves or muscle bellies of the residual limb can provide volitional control over analogous functions of the prosthesis. For example, electrodes positioned to sense contraction of wrist extensor muscles can trigger the force-generating component of the wrist extensor mechanism of the prosthesis. These electrodes can be integrated into the socket itself and sense myoelectric signals transcutaneously, similar to modern diagnostic electromyography devices. In another example implementation, implantable intramuscular and/or epimysial electrodes can be inserted into or adjacent to muscle bellies at the time of amputation or afterward, providing improved signal to noise characteristics and discrimination of signals intended for each muscle belly individually. A lead-wire traversing the soft-tissues to the skin surface or just beneath the skin surface can communicate these signals to the functional prosthesis, conferring volitional control by direct, transcutaneous, and/or subcutaneous communication. Complex volitional control can be designed to be grouped, repurposed, and/or context specific, providing an opportunity for preserved function in the absence of necessary muscles or nerves such as in extreme proximal limb amputees or amputation associated with significant damage to the residual body part. For example, volitional forearm pronation/supination while the elbow is flexed could execute a grasping/opening maneuver, and volitional forearm pronation/supination while the elbow is extended could execute a wrist abduction/adduction maneuver.

In some cases, anatomically analogous mechanical forces of a functional prosthesis act at a joint with multiple axes of motion in order to reproduce anatomic motion, thereby mimicking anatomic joints. Examples of joint motion include rotation, flexion, extension, abduction, adduction, and translation. An anatomic joint often includes articulating bones with cartilaginous surfaces and joint fluid that enable smooth motion and some degree of cushioning. Ligaments and joint capsules provide more rigid stabilization and limit freedom of motion about the joint axis, providing an anatomic range of motion. This range of motion can be reproduced using mechanical joints with analogous degrees of freedom, including ball-sockets, gimbals, cage-sockets, hinges, and other designs. Mechanical joints in a functional prosthesis may be internal to the surface morphology, and thus the ability to produce analogous mechanical function, in some cases, may outweigh the importance of biomimetic aesthetics. In a basic example, non-assembly articulating joints corresponding to various anatomic joints can be directly fabricated and subsequently integrated into a cable-pulley system or force-generating component at the joint itself, thus recreating anatomic motion. In some cases, for non-weight bearing body parts subject to smaller forces such as fingers and hands these types of joints may be preferred due to their size and simplicity. Increasingly complex joints subject to larger forces such as the knee and ankle can make use of more mechanically robust joints, several of which have been already been designed and manufactured (e.g., Jaipur knee/foot). These can be integrated into the design of a functional prosthesis subject to larger forces such as weight-bearing. The choice of mechanical joint and prosthesis complexity can be dictated by considerations such as cost, desired functionality, weight, and availability of materials including separately manufactured joints.

Although several design considerations are detailed above, these are merely illustrative examples. In practice, a prosthesis model can be generated based on an object model using some of the above described model design considerations in conjunction with any number of other considerations.

After a prosthesis model is generated, the process 100 continues by generating a set of instructions based on the prosthesis model (step 140). In an example implementation using 3D printing for fabrication, a prosthesis model can be converted into a set of printing instructions by using software specific to a destination printer and its materials, or by using freely available software (e.g., KISSlicer, Slic3r, and Cura).

As an illustrative example, printing instructions using widely used printer language such as G-code can be made prior to fabrication. A prosthesis model to be fabricated is analyzed for optimal orientation of fabrication, reducing the difficulty in fabricating architecture such as acute overhang angles, and ensuring the model fits in the printer-specific print bed volume. In some cases the model may exceed the print bed volume of a printer, a fact which may be amenable to considerations including selection of a different printer, piecemeal production of component parts, or separation of large components into suitably smaller components to be re-assembled subsequently. The prosthesis model may be de-convoluted in a contiguous slice-by-slice manner starting from an origin designated according to the model orientation, each slice corresponding to a point on the z-axis of the print bed volume, and each component of the slice corresponding to x and y coordinates on a Cartesian plane. There may be non-contiguous components on any given single slice, which may be supported by adjacent portions of a preceding slice, or independently with secondary support architecture that can be created in an automated or user-defined fashion. This secondary support architecture can be removed at the completion of fabrication, leaving an intact fabricated prosthesis model. Information within a set of printing instructions not corresponding to XYZ coordinates often include instructions for tool head speed, extrusion rate, and temperature, such as in the case of extrusion fused deposition modeling; or related to light wavelength and curing time such as in the case of digital light processing stereolithography (DLP-SLA). Thus printing instructions specific to the intended printer are made in such a way as to ensure correct fabrication of the prosthesis model.

In some implementations, printing instructions may not reflect a slice-by-slice de-convolution of a prosthesis model, and allow more direct and/or dynamic production of a prosthesis model. Printing instructions for certain fabrication techniques utilizing a tool head may be generated in such a way that the tool head is allowed to move with greater degrees of freedom (e.g., simultaneous motion about x, y, and z-axes), or generated in such a way that typically global parameters (e.g., extrusion rate, shell thickness, in the case of filament extrusion-based printing) can be defined locally within the prosthesis model. Generating printer instructions in this manner may be desirable in some cases according to the geometry of the prosthesis model and desired mechanical properties (e.g., direct fabrication of a spiral geometry of varying thickness that is more uniform and mechanically robust than an identical geometry fabricated in layers with a single extrusion rate and thus more susceptible to delaminating forces), and in other cases according to the printer mechanism (e.g., robotic arm-attached tool head compared to a tool head affixed to a stage and constrained to motion within a Cartesian plane). These instructions may also be encoded using printer language such as G-code, in some cases requiring additional processing within open-source CAD (e.g., Rhino3D with Project Silkworm plugin).

In some implementations, a set of instructions is generated based on only a portion of the prosthesis model. For instance, in some implementations, only portions of the prosthesis model that can be fabricated on a 3D printer are considered in order to generate instructions, while the other portions are ignored. For example, if the model contains a microcontroller, the microcontroller might be ignored during instruction generation, while structural elements supporting the microcontroller might be considered. In some implementations, multiple sets of instructions are generated for different portions of the prosthesis model. For example, two different sets of instructions might be generated for two different portions of the prosthesis model, such that each of the portions are fabricated separately.

Although example instruction generation implementations are described above, these are merely an illustrative example. In practice, instructions can be generated using other techniques, depending on the implementation.

This set of instructions is then executed using a 3D printer, causing the 3D printer to produce a prosthetic device for the patient (step 150). A 3D printer, or additive manufacturing (AM) device, is a device that can create a three-dimensional object based on an instruction set. As an example, FIG. 4D shows a completed prosthetic device printed by a 3D printer.

In some implementations, a 3D printer can create a three-dimensional object using one or more additive processes in which successive layers of material (e.g., liquid, powder, paper, hydrogel, or sheet metal) are laid down under computer control. Example processes can include extrusion-based techniques (e.g., for fused deposition modeling), wire-based techniques (e.g., for electron beam freeform fabrication), granular techniques (e.g., for direct material laser sintering, electron-beam melting, selective laser melting, selective heat sintering, and selective laser sintering), power bed and inkjet head 3D printer (e.g., for plaster-based 3D printer), lamination techniques (e.g., for laminated object manufacturing), living tissue-based techniques (e.g., hydrogel bioprinting) or light polymerized techniques (e.g., for stereolithography and digital light processing).

In an example implementation, upon executing a set of instructions, the 3D printer lays down successive layers of material (e.g., molten plastic, metal, or hydrogel) using a computer-controlled applicator (e.g., an extruder, adhesive inkjet, DLP projector). Each layer of material corresponds to a particular cross-section of the design model of the prosthesis. As each successive layer is laid down, the layer is joined or fused to its neighboring layers, creating a physical object having a thickness greater than each individual layer. Multiple layers are laid down and joined in this manner until a physical prosthesis is produced for the patient.

In some cases, after the 3D printer produces the prosthesis, the prosthesis can be directly used by the patient. In some cases, the prosthesis can be modified or refined (e.g., by the patient, a clinician, a caretaker, or some other person) prior to use by the patient. For example, a user might add additional structural, mechanical, and/or electrical elements to the prosthesis (e.g., actuators, microcontrollers, or other objects and materials, 3D printed or otherwise), remove portions of the prosthesis (e.g., by removing portions of the prosthesis that are not needed or meant to be temporary), or adjust the prosthesis (e.g., to fit adjustable portions of the prosthesis to better suit the user). In some implementations, a 3D printer might produce several physical objects, and the physical objects can be assembled and adjusted to form a prosthesis. In this manner, although the 3D printer is used to produce a prosthesis for the patient, the produced prosthesis can be subsequently modified and/or refined prior to actual use.

Although an example extrusion-based 3D printing process is described above, other types of 3D printing processes and 3D printing devices can be used, depending on the implementations. In some implementations, a 3D printer can include a commercially available consumer device (e.g. Formlabs Form 1, Kudo3D Titan 1, Type A Machines Series 1) or industrial device (e.g., EOS EOSINT P 800, NovoGen MMX Bioprinter, 3DSystems Projet).

As noted above, in some cases, the prosthesis model can be generated based on the object model, but need not be geometrically or mechanically similar. In some cases, the prosthesis model can be generally similar to the object model, but is not completely identical. This may be the case, in particular, if the prosthesis is not intended to fully replace a missing body part, but rather is intended to support or brace an injured body part. As an example, FIG. 4E shows a completed prosthetic device for a patient's hand. In this example, the prosthetic device acts as a brace for the patient's hand, and is not intended to fully replace the hand. Thus, the prosthesis device is not mechanically identical to the hand, but rather provides a protective support structure that surrounds portions of the patient's hand. Thus, a prosthetic device can be designed by first generating an object model of a patient's body part, then generating a prosthesis model based on, but not identical to, the object model.

As described above, imaging data from multiple imaging modalities can be used to generate an object model. The use of two or more different modalities can provide information that might otherwise be missing or difficult to ascertain using a single modality, and can be used to provide a more accurate object model for prosthesis design and fabrication.

Different combinations of imaging modalities can be used, depending on the implementation. For example, photographs or LiDAR data can be used to assess the exterior of a patient; this can be useful, for instance, in obtaining information regarding a patient's skin or the patient's posture. As another example, X-ray radiographs provide relatively high contrast between a patient's hard and soft tissues; this can be useful, for instance, in obtaining information regarding a patient's hard tissues (e.g., bones). As another example, MRI data can distinguish between different types of soft tissue; this can be useful, for instance, in obtaining information regarding specific tissues, organs, or other structures of interest within the patient. As yet another example, ultrasound data can visualize structures in motion; this can be useful, for instance, in obtaining information regarding a patient's beating heart (e.g., valves), pulsating vessels (e.g., aorta), or moving fetus (e.g., prenatal ultrasound). As above, these images can be segmented to extract the structure of interest. However, since each imaging modality provides potentially unique information, segmentation can be selectively performed on each set of images to extract specific structures from each of the sets of images. For example, structures pertaining to the exterior of the patient can be modeled from photographs or LiDAR data, structures pertaining to hard tissues can be segmented from CT images, structures pertaining to particular soft tissue can be segmented from MRI images, and structures pertaining to certain tissue in motion can be segmented from ultrasound images. These segmentations can be combined to generate a single composite 3D computerized model of the patient's anatomy, through a process of registration and transformation. During this processes, common features can be identified on each component model, for example identifying heart valve leaflets and their attachment to the valve annulus. Once identified, the component models can be transformed computationally such that each model is deformed until the registered points overlap with each other. If a component model is known to have higher spatial accuracy, for example a CT model in a multi-modality heart model including CT and ultrasound information, it may serve as a structural template onto which the transformation of the other models—in this case a heart valve model based on ultrasound imaging data—can be targeted. In this manner, the individual strengths of the imaging modalities are preserved and leveraged to produce a more complete and accurate composite model. In turn, this model can also be used to generate a physical model or prosthesis (e.g., using 3D printing).

As noted above, each imaging modality provides potentially unique information. For instance, CT data represent linear attenuation coefficients within a patient's body. Knowledge of the attenuation of varying tissues at various beam energies can be utilized for segmentation. As an example, bones may be highly attenuating, facilitating segmentation techniques including global thresholding, region growing, threshold painting, slice interpolation, edge identification, and atlas or shape-based identification. As another example, intravenous or other forms of administered contrast greatly increase blood pool and solid organ attenuation; thus contrast-enhanced studies can be similarly well suited for segmentation. As yet another example, soft tissue segmentation based on CT image data may also be robust, for example identifying tissues and minerals with specific dual-energy attenuation characteristics. In many cases, familiarity with diagnostic imaging anatomy is required, for example to identify geometries of interest within a narrow range of soft tissue attenuation values. CT image data are often isotropic acquisitions; thus segmenting geometries of interest can often provide representationally accurate volumetric information.

MRI data represent signal intensities within a patient's body that relate to quantum properties of hydrogen atoms and their immediate surroundings. Many MRI pulse sequences are available for a variety of diagnostic purposes, and knowledge of the sequences and the imaging characteristics of relevant anatomy and pathology on each sequence can contribute to successful, efficient, and accurate segmentation. As standard diagnostic protocols often call for specific sequences for specific purposes, knowledge of the sequences that are available on a routine study of a particular body region can also be useful. In some cases, MRI data can be isotropic; for example, MRI data can be acquired as an isotropic 3D data set. In some cases, MRI data can be in the form of 2D planar images; for example, MRI data can be acquired in 2D “slices” at various positions on the patient's body and according to various thicknesses. In some cases, 4D MRI data can be acquired to provide quantitative information related to blood flow within vessels. Protocols are often optimized to include specific sequences tailored for a diagnostic goal, often distinct from specific segmentation goals. Thus clear a priori intent to utilize image data for a specific segmentation goal (e.g., a particular type of tissue, organ, body part, or region) may provide additional opportunity to optimize image acquisition protocols, if this information is requested prior to image acquisition. These optimized sequences may include standard sequences with slight modification, standard sequences applied to non-standard body regions, sequences currently in development for research investigation, or novel sequences tailored for segmentation that provide little or no additional diagnostic information.

Ultrasound data represent echoes of sound waves transmitted through a patient's body. Ultrasound is often used, for example, for heart imaging or prenatal evaluations. Ultrasound images are often acquired and displayed in real-time, and thus can provide functional information. Volume information can be constructed from 2D acquisition images by registering them together as a “stack” (e.g., a series of images ordered according to their position on the patient's body). If position information of the ultrasound probe is known (e.g., through visual tracking or some other positioning system), representational accuracy of the volume can be improved. 3D ultrasound can also directly provide volume data by incrementally and automatically directing the ultrasound beam from within the transducer, facilitating construction of a 3D volume from 2D image data. These image data are often anisotropic; however, the resulting object models can be registered and transformed with respect to another object model made from image data with isotropic acquisition. For example, a 3D ultrasound of a heart valve can be made into an object model of the valve, which can be registered and transformed with respect to the valve annulus and leaflets on an object model of a heart based on CT data from an isotropic acquisition.

Photographic data represent incident visible light on a photosensitive medium. Thus, photographic data can depict visible surface appearance of a patient's body. The process of photogrammetry entails acquiring circumferential photographic data from multiple closely adjacent camera positions, maintaining similar exposure and focus point. The images can be analyzed for common features, and based on the relative positions of these common features and the parallax between images, camera position can be inferred computationally and pixel information can be redistributed in 3D space (e.g., a “point cloud”). A point cloud can then be used to construct a 3D object model, suitable for further manipulation in CAD. Additionally, because the point cloud contains pixel color information, a texture map demonstrating the surface detail in color can be made and applied to the model.

In an example implementation, an object model can be generated based on photographs and X-ray radiographs in order to visualize features of the body part that might otherwise be hard to visualize using a single imaging modality; for example, photographs can provide information regarding the outer surface of the body part, while the X-ray information can provide information regarding the internal structure of the body part. In another example, CT and MRI data can provide additional three-dimensional anatomic information and can similarly provide information that might otherwise be difficult to ascertain using other imaging modalities. For example, soft tissue swelling, contusion, and/or laceration injury that might be difficult to visualize using X-ray imaging can be better imaged using MRI. Collectively, this information can be used to further improve the model design. For instance, force-bearing surfaces on a prosthetic device can be designed in deliberate avoidance of soft tissue pathology, or constructed in such a way as to provide a removable or otherwise accessible window for dressing change and/or administration of treatment such as topical therapy. Patient comfort can also be greatly enhanced by preserving the ability to address areas of skin irritation. In a similar fashion, the prosthesis can be designed such that zones of adjustable fit are integrated into areas of the prosthesis where a lesser degree of swelling is anticipated, resulting in increased patient comfort. Further, by designing the prosthesis such that it avoids or accommodates regions of swelling, the resulting prosthesis can be used by the patient more quickly after an injury. For example, if the patient has a broken arm, a prosthesis can be designed such that it avoids regions of swelling of the arm, such that the prosthesis can be fitted shortly after injury, without first waiting for the swelling to subside. Patient comfort may be further enhanced through the use of waterproof or submersible materials, facilitating daily activities such as showering.

In many cases, after an injury to a limb, stabilizing and positioning the limb properly is important for both patient comfort and effective healing. While diagnostic medical imaging is often obtained deliberately in the desired position for healing, this cannot always be presumed, particularly in the acute setting. In contrast, photographs taken of a patient subsequent to clinical stabilization (e.g., post-reduction) for the purpose of prosthesis modeling can be obtained to reflect the desired positioning of the injured body part (e.g., from a comfort standpoint, healing standpoint, or both). For example, neutral limb positioning may be desired for healing, however associated medical imaging may be obtained in another position in the acute setting. During image data acquisition for the purpose of prosthesis modeling, the limb may be appropriately positioned for optimal healing. Thus, an object model can be generated in a manner that reflects the desired positioning of the body part. Further, in some implementations, object models can be manipulated and re-positioned virtually in CAD software (e.g., Solidworks, Netfabb, 3-MaticSTL) to additionally optimize positioning. Further, this can provide an opportunity to simulate force distribution in response to variable forces through techniques such as finite element and tolerance analysis of both the limb and prosthesis, as described above.

In an illustrative example, an implementation of process 100 is used to produce a prosthesis for a patient suffering from a distal radius fracture, a common forearm injury. After the patient's acute presentation is addressed in a medical setting, the forearm is stabilized (e.g., set in a position that maintains patient comfort and facilitates healing), and images are obtained of the arm. For example, the patient's forearm can be photographed from a variety of angles (e.g., using a digital camera to capture photographs from different perspectives relative to the arm). As another example, the patient's arm can be imaged using X-ray imaging. The acquired images are then used to create an object model of the patient's forearm. Each image can contribute to generation of the object model. For example, the photographs can be used to ascertain the surface of the patient's arm and to determine the overall volume of the arm. The X-ray radiographs can be used to determine the location of bones and the nature of the fracture. This information can be used, for example, to determine instability in the radius caused by the fracture, predict the direction of the instability, and determine structural elements that can be used to stabilize the radius (e.g., by implementing structural elements in the prosthesis that maintain close apposition of the fracture fragments). Points of stability on the uninjured proximal radius, the adjacent ulna, and distal carpal and hand bones can also be identified and taken advantage of as areas better suited for force distribution and stabilization. Knowledge of classic and variant anatomy can be used to further inform prosthesis design and, for example, avoid known areas where compression of sensitive neurovascular structures is likely to occur, and/or optimize positioning for healing and comfort which may be specific to a particular anatomic configuration. Based on this object model and the predicted interaction between each of the intact and injured components of the forearm, a design for a stabilizing prosthesis can be generated to stabilize and support the forearm. The mechanical robustness of the prosthesis and the nature of its interaction with the object model may be examined in a simulated environment, for example, by the methods described above.

Although photography and X-ray radiography are described above, these are merely illustrative examples. In some implementations, CT and MRI data can also be obtained, either in addition to or instead of photographs and X-ray radiographs. Similar object models can be generated using these imaging data, and a prosthesis can be similarly designed using this object model.

In some cases, a model of a patient can be combined with a model of a piece of surgical hardware or a medical device. This can be useful, for example, in simulating the interaction between the two. As an example, a model of a piece of surgical hardware or a medical device can be virtually applied to a patient object model that has been converted into a finite element mesh, and the dynamic interaction between the two can be simulated using finite element analysis (FEA). Mesh simplification, such as mesh decimation, may be used to facilitate computation. As another example, finite element meshes of vascular structures can be made prior to procedures for altering a patient's hemodynamics, and the flow of blood within these structures can be simulated using computational fluid dynamics (CFD). The mesh may be manipulated to approximate the result of the planned procedure, enabling simulation of post-operative alteration of hemodynamics. Similarly, mesh simplification, such as mesh decimation, may be used to facilitate computation.

In some cases, the object model can serve as a template against which another model is designed or customized (e.g., a socket for a prosthetic limb). An object model of a socket can be manipulated to fit the contour of the limb object model. In some cases, this may entail registration and transformation of the socket object model with respect to the limb object model, Boolean subtraction at the limb-socket interface, or other techniques to integrate surface contour information regarding the patient onto the object model of the intended device.

Implementations of this process allow for the fabrication of a prosthesis without physical prototyping. For example, instead of fabricating a prototype and iteratively revising the prototype until it suits the patient's needs, a prosthesis can instead be designed based on an object model, and the design can be revised as necessary prior to fabrication. If significant changes in body part morphology are suspected, images of the body part may be re-acquired in order to construct an object model reflecting the morphologic change, which may subsequently inform revision of the prosthesis model in an analogously iterative process. Likewise, as the prosthesis is designed specifically for a particular patient, the prosthesis can be designed in such a way that it reduces the number of superfluous structural elements that might otherwise not be needed to support the specific injury to the body party. Thus, a prosthesis can be fabricated to be more open compared to, for example, a plaster or fiberglass cast (such that patient comfort is increased), while maintaining the structural elements needed to adequately stabilize and support the injured body part. In cases where revision of the fabricated prosthesis is desired, information relating, for example, to its suboptimal function or to patient discomfort, may be integrated in a feedback loop at any point in the generation of the object and/or prosthesis model and its fabrication. While one aim of this method is to avoid the need for this type of post-fabrication revision, the advantages of the process allow for rapid revision and fabrication in cases where it is desired.

Design and fabrication of a prosthesis using a methodology allowing for rapid revision such as this has further benefits in pediatric and adolescent populations, where growth and maturation result in rapidly changing morphology over time, often requiring multiple unique prostheses. Scoliosis, for example, may in certain cases be treated with serial casting and bracing, making use of multiple unique prostheses tailored to the changing patient size, morphology, and eventual morphologic goal. To illustrate further, congenital deformity such as clubfoot may be corrected non-operatively by methods similar to the Ponseti method, making use of several unique prostheses tailored to the desired degree and form of limb manipulation at a particular point during the process of deformity correction. Adjustable and/or modular components can further improve ease of and control over the degree of manipulation and frequency of adjustment, and removable components can enable more frequent and facile evaluation of the progress of correction, forgoing time consuming and costly removal of traditional plaster casts to check progress and further adjust the degree of manipulation. The use of waterproof or subermisble materials in such cases is also of particular benefit, allowing for daily activities such as bathing, which are otherwise difficult with the use of traditional materials such as plaster.

Although process 100 has been described above in the context of producing prostheses, the process 100 can also be adapted for the production of clinical models. For example, to produce a purely computerized model of a patient, step 110 (obtaining imaging data) and step 120 (generating an object model) can be performed, and step 130 (generating a prosthesis model), step 140 (generating a set of instructions), and step 150 (executing the set of instructions) can be skipped. As another example, to produce a physical model of a patient, step 110 (obtaining imaging data) and step 120 (generating an object model) can be performed to produce a computerized model of the patient, and the computerized model obtained in step 120 can be directly used in step 140 (generating a set of instructions), and step 150 (executing the set of instructions). In this manner, each of the steps of the process 100 can be selectively performed, depending on the desired end product.

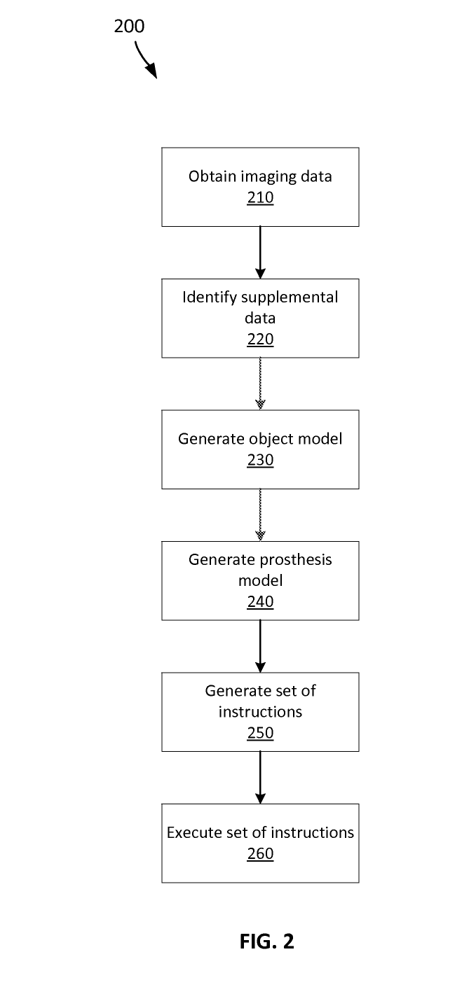

In the implementations described above, the object model of the patient's body part and the corresponding model or prosthesis are designed based only on information pertaining to the patient, including the patient's intact contralateral anatomy. That is, images are obtained for a particular patient, and a model or prosthesis is fabricated based on these images. In some implementations, information regarding other patients can also be used in generating the object model and prosthesis design. An example implementation of a process 200 for fabricating a model or prosthesis using information regarding multiple patients is shown in FIG. 2.

The process 200 begins by obtaining imaging data corresponding to a body part of a patient (step 210). Step 210 can be similar to step 110, as described above. For example, one or more different types of imaging data can be obtained, including photographs, computed tomography (CT) data, magnetic resonance imaging (MRI) data, ultrasound data, X-ray radiographs, among others.

After imaging data is obtained, the process 200 continues identifying supplemental data corresponding to one or more other patients (step 220). Supplemental data can include, for example, imaging data, object models, or prosthesis models corresponding to one or more other patients. In some cases, supplemental data can be used when certain imaging data might otherwise be unavailable for the present patient. For example, during the course of treatment, the present patient (or his caretaker) might have access to a camera, but might not have access to X-ray, CT, ultrasound, or MRI equipment. In this case, supplemental data can include X-ray radiographs, CT data, ultrasound data, and/or MRI data of other patients to supplement the present patient's photographs. In some cases, supplemental data can be used to supplement imaging data that is already available for the present patient. For example, during the course of treatment, the present patient (or his caretaker) might have full access to a camera, as well as to X-Ray, CT, ultrasound, and MRI equipment. In this case, supplemental data can include photographs, X-Ray radiographs, CT data, ultrasound data, and/or MRI data of other patients to further supplement the information that is available for the present patient. For instance, supplemental data might include images of a higher resolution than what is currently available for the present patient, images taken from additional perspectives not available for the present patient, MRI sequences tailored to visualize certain tissue types which were not acquired for the present patient, or other types of imaging data that can be used to supplement imaging data that is already available. In some circumstances (e.g., when the present patient is badly injured or is partially or completely missing a body part and unable to make use of contralateral anatomy), the supplemental data can provide surrogate information regarding the missing or badly injured body part.

Supplemental data can be identified in a variety of ways. In some implementations, supplemental data can be identified by determining other patients that have similar characteristics as the present patient. For instance, in designing a prosthetic limb for the present patient, other patients with limbs having similar physical characteristics (e.g., similar shapes, spatial dimensions, volumes, or other characteristics) can be used as potential sources for supplemental information. As an example, if photographs of a particular limb are available for the present patient, photographs other patients' limbs can be reviewed in order to find similarities. If a similar other patient is located (e.g., a patient having a limb with similar physical characteristics as the present patient), the photographs of the similar patient can be used as supplemental information. In addition, additional imaging data associated with the similar patient (e.g., X-ray radiographs, CT data, or MRI data) can also be used as supplemental information.

As another example, if an object model is already available for the present patient's body part, the object model can be compared to those of other patients to determine potential sources for supplemental information. If similar object models (e.g., object models depicting a body part with similar characteristics) are located, these object models can also be used as supplemental information, as well as additional imaging data associated with the similar patient (e.g., X-ray radiographs, CT data, ultrasound data, or MRI data).

Body parts can have similar physical characteristics if they are relatively similar to each other with respect to one or more metrics. As described above, limbs having similar physical characters might have similar shapes, spatial dimensions, volumes, or other characteristics. For example, body parts having similar shapes might have outer surfaces that are similar to each other. It is possible, for instance, to compare quantitatively the 3D morphology of analogous body parts from two patients by registering surface landmarks that are stereotypical to human anatomy. A transformation function can be generated to express the necessary distortion required to map one model onto the other by example methods such as affine transformation or optimal mass transport; the magnitude of this transformation serves as an indication of fit. As another example, body parts having similar spatial dimensions might have one or more corresponding spatial dimensions that are similar (e.g., having lengths or widths within a particular percentage of each other, such as 5%, 10%, 15%, 20%, and so forth). This data can be obtained from existing anatomic atlases, or can be derived from image databases where morphologic information exists on a large scale (e.g., on a population scale). For example, in many cases, a single dimensional characteristic of the radius, such as its width across the physis or “growth plate,” can be expected to correlate with other spatial dimensions of the bone, and with spatial dimensions of other bones. In another example, general biometric characteristics such as height and weight can be expected to correlate with dimensions of specific body parts such as forearm length and wrist circumference. The known or calculated statistical strength of these correlations can be used to assign relative weights to multiple dimensional measurements when comparing two patients, and can provide an estimation of the degree and range of fit regarding the accuracy of candidate surrogate anatomy. As another example, body parts having similar volumes might have component volumes that are similar (e.g., having volumes within a particular percentage of each other, such as 5, 10%, 15%, 20%, and so forth). For example, vertebral bodies can be found to have variable volumes of mineralized bone, even for relatively similar morphologic volumes. Similarly, the volume of cortical bone within similarly sized forearms can be variable according to the contribution of soft tissue such as subcutaneous fat to the overall forearm volume. As examples, these differences can arise from age-related changes, differences in systemic states of osseous mineralization, nutritional states, or use-related changes, among other factors. This may be particularly relevant when load-bearing body parts are under examination, as structural changes related to load-distribution over time may not be well represented by either single-dimension or surface morphology alone. As further example, this may be particularly relevant when body parts under examination lie at the extremes of nutritional states such as obesity or malnourishment, as surface morphology may be disproportionately representative of certain component volumes such as muscles, subcutaneous fat, or bones. Although individual metrics are described above, other metrics can also be used to determine the physical similarity between two or more body parts. Further, in some implementations, multiple metrics can be used in conjunction to determine physical similarity.

In some implementations, other information can also be used to determine potential sources for supplemental information. For example, patients can be filtered on the basis of demographic information (e.g., gender, age, ethnicity, and so forth) or general physical attributes (e.g., weight, height, or other physical attributes) in order to determine potential sources for supplemental information.

In some implementations, multiple criteria (e.g., demographic information, general physical attributes, imaging data, and/or object models) can be used to determine potential sources for supplemental information. The importance of each individual criterion in determining sources for supplemental information can vary, depending on the application. In some implementations, potentially similar supplemental data can be first presented to a user for manual confirmation before being used in subsequent processing.

As an example, a limb or body part surface model generated by photogrammetry could be considered a common origin for identification of supplemental anatomic information. Surface landmarks can be identified at the time of image acquisition or based on the 3D object model, and information regarding scale may also be integrated into the image acquisition phase. Standard demographic identifiers can also be collected directly, as well as biometric data such as height, weight, gender, etc. From this data set, image data from other patients containing potential supplemental information can be cross-matched at multiple levels (demographically, morphologically, biometrically etc.) according the described goodness-of-fit estimations. As already described, the near-mirror symmetry of human anatomy allows reliably accurate supplemental information to be accessible from preserved contralateral anatomy, within the same patient or regarding the contralateral side of other patients. This can be useful in some cases, for example when this information is already in existence (e.g., prior studies of the opposite side) or otherwise easily obtained (e.g., according to facility access). Thus, for unilateral amputees, a preserved contralateral limb can provide an excellent template upon which a device such as a prosthesis can be designed (e.g., a template to model a socket or other feature of the body part). In some cases, this data is lacking and additional supplemental data is required, such as in the case of bilateral amputation or unilateral amputation with severe contralateral injury. In these cases, morphologically similar supplemental data can be identified by the above methods, including use of demographic identifiers, general physical characteristics, morphologic information and/or object models of intact but not specifically relevant anatomy (e.g., physical characteristics of a foot regarding a present patient requiring a hand prosthesis, used to identify supplemental hand information from other patients with similar foot characteristics) to further refine the search.

Multimodal image data of the body part of interest can also be integrated, as available and as warranted by the specific application. These may provide redundant information or information of variable accuracy. For example, in many cases, radiography provides the highest spatial resolution of bony anatomy, allowing for accurate identification of acute fractures and allowing for design considerations relevant to the fit, stabilization, and comfort of a brace. This information, however, is often provided in two dimensions, and may be subject to projectional distortion/magnification according to the position of bony anatomy relative to each other and to the beam source/receptor. Thus, while radiography is a useful adjunct to surface morphology acquired by photogrammetry, radiography alone may be insufficient in some cases to generate a 3D object model. CT, by contrast, can provide volumetric soft tissue and bone image data that is highly representational of true morphology, and can in some instances provide sufficient information alone to construct an object model. When surrogate CT data is desired, for example in designing an extremity prosthesis in a setting without access to cross-sectional imaging, candidate supplemental data can be searched for on multiple levels, as already described. In an example case of unilateral amputation, an object model of the intact contralateral limb may be obtained by photogrammetry, and may be used as a comparison against which candidate supplemental CT data can be evaluated by morphologic comparison. After a suitably matching CT data set is identified, it may be transformed so that the surface contours closely resemble the surface morphology of the intact contralateral limb of the present patient, and then mirrored to reflect the amputated limb. Thus, supplemental CT data of an amputated limb that is matched to the present patient can be made available in a setting where access to cross-sectional imaging is difficult or lacking, providing some of the benefits of CT morphologic information where it would otherwise not be available. A similar matching process and search for supplemental MRI data may be desired in some cases as well, for example in cases where particular soft tissue anatomy is of primary concern, such as in the design of a functional prosthesis or prosthesis socket.

In an example case of bilateral amputation, supplemental CT data can be identified according to determinable patient characteristics such as morphology of existing intact anatomy, demographic identifiers, height, weight, age etc. For clarity, a bilateral leg amputee of a certain age, weight, and height, living in a specific geographic region, with a certain wrist circumference; may be able to use as supplemental data the imaging data from the leg of another person of similar age, weight, and height living in a similar geographic region, with a comparable wrist circumference. This supplemental data can be used to generate an object model. Thus, supplemental CT data that is matched to the patient in the absence of directly analogous anatomy can be made available in a setting where access to cross-sectional imaging is difficult or lacking, providing the benefits of CT morphologic information where it would otherwise not be available. A similar matching process and search for supplemental MRI data may be desired in some cases as well, for example in cases where particular soft tissue anatomy is of primary concern, such as in the design of a functional prosthesis or prosthesis socket.

Over time, this process can yield sufficient insight into the reliably differentiating characteristics of human anatomy on a population scale, thereby informing the construction of representational atlases according to those scales of most reliable morphologic, demographic, biometric, and other categories of differentiation, thereby improving the efficiency and accuracy of identifying supplemental image data. Although the applications of such a database herein relate to design and fabrication of a prosthesis, similar applications of such methods as those described can be easily imagined in the fields of forensics and anthropology, for example.

After supplemental data is identified, the process 200 continues by generating an object model corresponding to the body part based on the imaging data and the supplemental data (step 230). Step 230 can be similar to step 120, as described above, except that supplemental data is also used to generate the object model. For example, photogrammetry can be used to extract surface points from a sequence of photographs. Further, X-ray radiographs, CT data, ultrasound data, and/or MRI data can be used to generate an object model.

After the object model is generated, the process 200 continues by generating a prosthesis model based on the object model (step 240), generating a set of instructions based on the prosthesis model (step 250), and the set of instructions is executed using a 3D printer, causing the 3D printer to produce a prosthetic device for the patient (step 260). Steps 240, 250, and 260 can be similar to steps 130,140, and 150, respectively, as described above.

Similarly, although process 200 has been described above in the context of producing prostheses, the process 200 can also be adapted for the production of clinical models. For example, to produce a purely computerized model of a patient, step 210 (obtaining imaging data), step 220 (identifying supplemental data), and step 230 (generating an object model) can be performed, and step 240 (generating a prosthesis model), step 250 (generating a set of instructions), and step 260 (executing the set of instructions) can be skipped. As another example, to produce a physical model of a patient, step 210 (obtaining imaging data), step 220 (identifying supplemental data), and step 230 (generating an object model) can be performed, and the computerized model obtained in step 230 can be directly used in step 250 (generating a set of instructions), and step 260 (executing the set of instructions). In this manner, each of the steps of the process 200 can be selectively performed, depending on the desired end product.

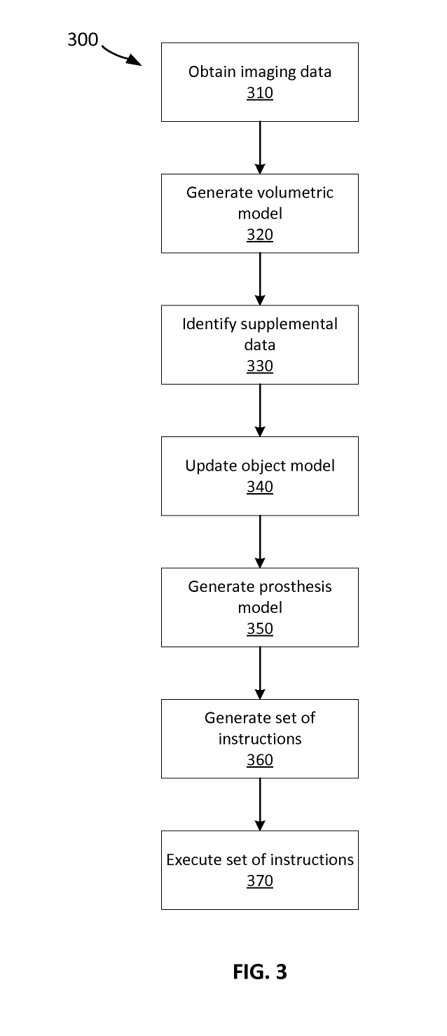

In the example implementation described above, supplemental data is identified before the object model is generated; after supplemental data is identified, the imaging data and the supplemental data are collectively considered in generating the object model. However, this need not be the case. For example, in some implementations, an object model can be generated based on the imaging data of the present patient, where relevant anatomy is intact. Supplemental data can then be used to update the object model or generate a new object model. An example implementation of a process 300 is shown in FIG. 3.

The process 300 begins by obtaining imaging data corresponding to a body part of a patient (step 310). Step 300 can be similar to steps 110 and 210, as described above. For example, one or more different types of imaging data can be obtained, including photographs, computed tomography (CT) data, magnetic resonance imaging (MRI) data, X-ray radiographs, among others.

After imaging data is obtained, the process 300 continues by generating an object model corresponding to the body part based on the imaging data (step 320). Step 320 can be similar to step 120, as described above. For example, an object model (including volume, surface, solid-body, and/or dynamic information) can be generated based on photographs (e.g., photographs analyzed using photogrammetry), X-ray radiographs, CT data, ultrasound data, and MRI data.

After generating an object model, the process 300 continues by identifying supplemental data corresponding to one or more other patients (step 330). Step 330 can be similar to step 220, as described above. For example, one or more criteria (e.g., demographic information, general physical attributes, imaging data, and/or object models) can be used to identify potential sources for supplemental information. In addition, as an object model has already been generated based on the imaging information, the generated object model can be used to identifying supplemental information. For example, an object model generated using the imaging data can be compared against object models of one or more other patients according to demographic information and other attributes. In some implementations, the comparison between the object model of the present patient and those of the other patients can be expressed as a functional transform. Transformational techniques such as affine transformation or optimal mass transport can be utilized for the body part of interest to express the degree of distortion required to match one model to another. The relative magnitude of transformation required can act as a measure of morphologic similarity, and adequately similar object models can then be identified as supplemental data. Further, radiographic information, CT data, ultrasound data, and MRI data associated with that object model can also be used to identify potential supplemental data. As above, in some implementations, potentially similar supplemental data can be first presented to a user for manual confirmation before being used in subsequent processing.

After identifying supplemental data, the process 300 continues by updating the object model based on the supplemental data (step 340). In some implementations, updating the object model can include re-generating the object model based on the both the imaging data and the supplemental data (e.g., as described in reference to step 230 above). In some implementations, updating the object model can include transforming the object model based on the supplemental data. For example, inclusion of radiographic data into an object model acquired by photogrammetry may reveal exaggerated bony protuberances that are partially masked on a surface model by a corresponding thinning of overlying subcutaneous tissue. This can be reconciled by local adjustments to the object model in the area of the discrepancy, potentially improving the prosthesis fit and patient comfort, especially in force bearing areas and areas of desired stabilization. In some implementations, updating the object model can include discarding the object model and generating a new object model using only the supplemental data. For example, while a morphologically accurate surface model can be generated from an intact contralateral limb in the setting of unilateral amputation, it may be preferable to discard such a model if volumetric data of the previously intact limb is available, such as a pre-operative CT of the relevant limb.

After the object model is updated, the process 300 continues by generating a prosthesis model based on the object model (step 350), generating a set of instructions based on the prosthesis model (step 360), and the set of instructions is executed using a 3D printer, causing the 3D printer to produce a prosthetic device for the patient (step 370). Steps 350,360, and 370 can be similar to steps 130,140, and 150, respectively, as described above.

Similarly, although process 300 has been described above in the context of producing prostheses, the process 300 can also be adapted for the production of clinical models. For example, to produce a purely computerized model of a patient, step 310 (obtaining imaging data), step 320 (generating a volumetric model), step 330 (identifying supplemental data), and step 340 (updating the object model) can be performed, and step 350 (generating a prosthesis model), step 360 (generating a set of instructions), and step 370 (executing the set of instructions) can be skipped. As another example, to produce a physical model of a patient, step 310 (obtaining imaging data), step 320 (generating a volumetric model), step 330 (identifying supplemental data), and step 340 (updating the object model) can be performed, and the computerized model obtained in step 340 can be directly used in step 360 (generating a set of instructions), and step 370 (executing the set of instructions). In this manner, each of the steps of the process 300 can be selectively performed, depending on the desired end product.

In some implementations, identifying supplemental data and updating the object model can be optional. For example, upon generating an object model, a determination can be made if the object model is acceptable. This determination can be made subjectively by visual inspection and/or prior experience, or on a quantitative basis with thresholds of acceptability. Markers of scale, for example, can be re-examined on a completed object model to confirm appropriate scaling. Additional dimensional measures can provide adjunctive points for quality assessment. For example, a distal upper extremity model constructed by photogrammetry using a linear scale applied along the axis of an intact forearm can be quality checked by comparing known forearm circumferences at several levels along the scale marker (measured at the time of acquisition) against circumference measures at corresponding levels on the digital object model. When agreement is calculated above a particular threshold, the object may be considered to have sufficient accuracy, and the object model is used to generate the set of instructions. If not, supplemental data is identified or primary data is re-acquired in order to update the object model. If a fabricated prosthesis is determined to be ill-fitting or poorly functioning, the nature and degree of sub-optimal performance can be ascertained, for example, by verbal description or direct indication upon the prosthesis, such that rapid revision of the object and/or prosthesis model may proceed with this information feeding back at any point in the above process determined to be the most useful and relevant point of entry for reprocessing.

In an illustrative example, an implementation of process 300 is used to produce a prosthesis for a patient suffering from an elbow fracture. In this example, the patient does not have access to CT or MRI equipment, but has access to a camera and X-ray equipment. Photographs and X-ray radiographs of the patient's elbow are obtained, and are used to generate an object model for the elbow. After generation of the object model, the patient's demographic information can be used to identify a pool of demographically similar other patients (e.g., as described above), and the present patient's object model can be used to identify candidates from the pool who have a similar elbow. As an example, morphologic characteristics of the present patient's distal humerus, proximal radius and ulna, trochlea, and olecranon can serve as reference comparisons against which potential candidates can be judged. Once one or more adequately similar candidates are identified, supplemental imaging data associated with the similar candidates (e.g., CT data and MRI data) can be obtained, and an updated object model can be generated (e.g., using the techniques described above) taking into consideration the surrogate CT and/or MRI data of the other similar patients. This updated object model now includes information that would otherwise be difficult to obtain without the supplemental information. For example, the updated object model might contain more detailed information regarding the elbow's soft tissue, which may not have been clearly visible in the X-ray radiographs. Further, as the updated object model contains more information regarding the elbow, this new information can be used to develop dynamic models that more accurately describe the interaction between each of the components of the patient's elbow (e.g., the interaction between each of the bones and soft tissue). Further, the present patient's information (e.g., his demographic information, photographs, and X-ray radiographs) can be added to the pool of candidates for potential use as supplemental data for other patients.

As described above, in some cases, an object model of a patient can be produced using imaging data from multiple different imaging modalities. The use of two or more different modalities can provide information that might otherwise be missing or difficult to ascertain using a single modality, and can be used to provide a more accurate object model for prosthesis design and fabrication.

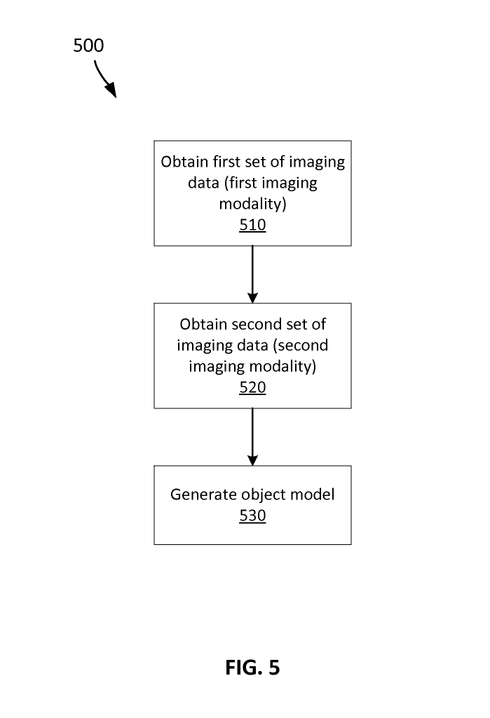

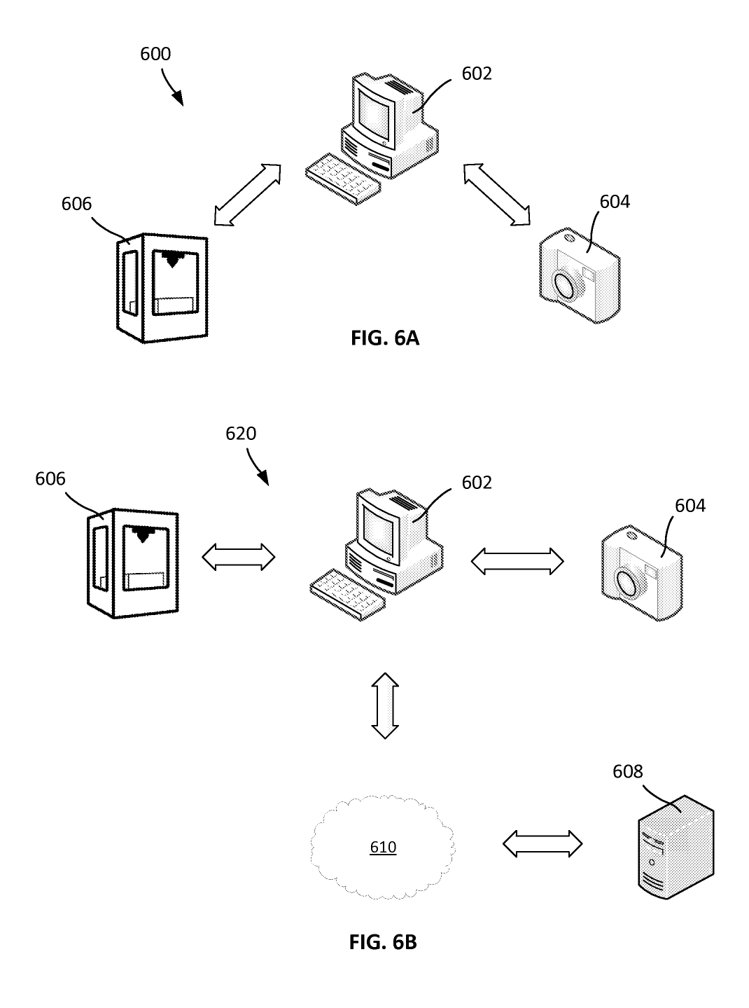

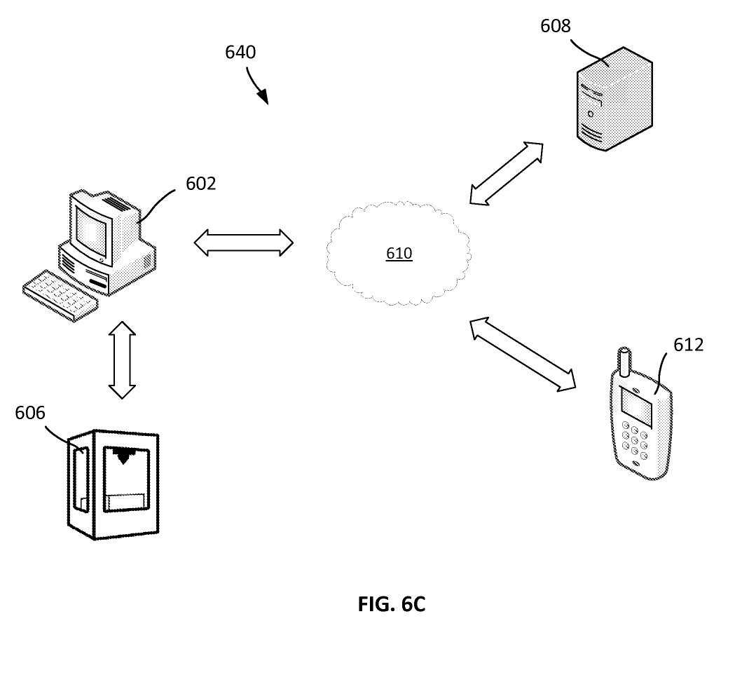

An example process 500 for producing an object model of a patient using multiple different imaging modalities is shown in FIG. 5.

The process 500 begins by obtaining a first set of imaging data corresponding to a body part of a patient, where the first set of imaging data was acquired using a first imaging modality (step 510). Step 510 can be similar to step 110, as described above. For example, the first set of imaging data can be obtained using an imaging modality such as photography, computed tomography (CT), ultrasound, magnetic resonance imaging (MRI), X-ray, among others.

After the first set of imaging data is obtained, the process 500 continues by obtaining a second set of imaging data corresponding to the body part of the patient, where the second set of imaging data was acquired using a second imaging modality different than the first imaging modality (step 520). Step 520 can be similar to step 110 or 510 as described above. For example, the second set of imaging data can be obtained using an imaging modality such as photography, computed tomography (CT), magnetic resonance imaging (MRI), X-ray, among others.

After the first set of imaging data is obtained, the process 500 continues by generating an object model corresponding to the body part based on the first set of imaging data and the second set of imaging data (step 530). Step 530 can be similar to steps 120, as described above. For example, an object model (including volume, surface, solid-body, shape, dimension, and/or dynamic information) can be generated based on photographs (e.g., photographs analyzed using photogrammetry), X-ray radiographs, CT data, ultrasound data, and MRI data.

In some cases, the object model can be generated by identifying portions of the body part from the first set of imaging data and identifying different portions of the body part from the second set of imaging data and generating a composite set of data based on the identifications. In some cases, the portions of the body part from the first set of imaging data correspond to different tissue types than the portions of the second body part. As examples, different tissue types can include bone, connective tissue, vascular tissue, muscle tissue, nerve tissue, and epithelial tissue. In some cases, the portions of the body part can be manually identified (e.g., by a human reviewer).

As described above, in some cases, the portions of the body part identified from the first set of imaging data have higher image contrast using the first imaging modality than the second imaging modality. In other cases, the portions of the body part identified from the second set of imaging data have higher image contrast using the second imaging modality than the first imaging modality. For example, a particular portion of the body part identified using one modality (e.g., CT) may have higher image contrast than that portion of the body part using another modality (e.g., ultrasound). Thus, the body part can be identified from the set of imaging data that provides the best, or otherwise sufficient, level of image contrast to distinguish the body part from the surrounding tissue.

As described above, in some cases, generating the object model can include identifying one or more anatomical structures from each of the sets of image data. Anatomical structures can be identified, for example, by segmenting the sets of imaging data based on one or more properties of the first set of imaging data. These properties can include, for example, patterns of localized image intensity, localized image contrast, and/or localized geometric shape.

As described above, in some cases, generating the object model can include registering the first set of imaging data and the second set of imaging data to a common geometric space. As examples, the first and second sets of imaging data can be registered based on a location of one or more common body part features in the first and second sets of imaging data.

As described above, in some cases, the object model can be used to generate a set of instructions for a three-dimensional printer based on the object model, such that the three-dimensional printer produces a physical model or prosthesis.

### Example Applications

Implementations of these techniques can be used in a variety of clinical applications.

To illustrate, Table 1 shows example clinical specialties where implementations of the described techniques can be applied. As described above, in some cases, clinical models can be generated based on medical image data obtained using multiple different imaging modalities (e.g., two, three, four, or more different imaging modalities). The use of two or more different modalities can provide information that might otherwise be missing or difficult to ascertain using a single modality, and can be used to provide a more accurate model or prosthesis. Table 1 shows example combinations of imaging modalities that can be used in particular contexts.

TABLE 1
Example applications of the disclosed techniques.

| Specialty | Procedure/Model | Modalities | Example Implementation |
|---|---|---|---|
| Biomedical engineering | Bioink and bio- | CT, MR, | Bio-compatible device design, direct |
| | compatible printing | US, PX | tissue printing |
| | Organ disease modeling | CT, MR, | Living tissue (macro and |
| | | US, PX | microscopic) models of disease and |
| | | | disease progression |
| Cardiology | Pulmonary vein ablation | CT, US, | CT for structure, US for valves, |
| | | MR | MR/US for functional assessment |
| | ASD/VSD repair | CT, MR | Virtual and ex vivo device sizing |
| | Appendage occlusion | CT | Virtual and ex vivo device sizing |
| | Valve repair | CT, MR, | CT for structure, US for valves, |
| | | US | MR/US for functional assessment |
| Cardiothoracic surgery | Superior sulcus tumor | CT, MR, | Tumor morgins, brachial plexus |
| | resection | PX, US | involvement, surgical approach |
| | | | planning |
| | LVAD implantation | CT, MR | Device fitting and flow modeling |
| | Heterotopic, orthotpoic | CT, MR | Bypass/transplant planning |
| | heart transplant | | |
| | Tracheobronchial | CT, MR | Airway modeling and custom stent |
| | stenting | | design |
| | Valve repair | CT, MR, | CT for structure, US for valves, |
| | | US | MR/US for functional assessment |
| Neurology/Neurosurgery | Transphenoidal resection | CT, MR | Sinus architecture, tumor modeling, |
| | | | neurovascular modeling |
| | Suboccipital resection | CT, MR | Calvarial architecture, tumor |
| | | | modeling, neurovascular modeling |
| | Craniotomy | CT, MR | Calvarial architecture, device |
| | | | modeling |
| | Spinal | CT, MR | Vertebral cage modeling, |
| | fusion/stabilization | | laminectomy planning, |
| | Cranial nerve modeling | CT, MR | Facilitate diagnosis, surgical |
| | | | planning |
| Orthopedic surgery | Internal fixation | CT, XR, | CT for bones, MR for soft tissue |
| | | MR | e.g., cartilage/ligaments/tendons/ |
| | | | nerves |
| | External fixation/cast | CT, XR, | Surface contour for custom cast |
| | | PX, MR | modeling |
| | High tibial osteotomy | CT, MR | Preoperative assessment + cutting |
| | | | guide modeling |
| | Resection | CT, MR | Cutting guide + filler modeling |
| | Osteochondral repair | CT, MR | Donor site, repair site, cutting guide |
| | | | and pedicle modeling, kinematics |
| Otorhinolaryngology | Cochlear implant | CT, MR | Middle/inner ear architecture, |
| | | | neurovascular modeling |
| | Auricle reconstruction | CT, MR | External ear architecture, implant |
| | | | modeling |
| | Nasal reconstruction | CT, MR | Nasal bone/cartilage and septum |
| | | | modeling. |
| | Sinonasal surgery | CT, MR | Sinus drainage pathway modeling, |
| | | | mucosa modeling, airway |
| | | | modeling/device design |
| Pediatric surgery | Developmental hip | MR, US, | Surface contour modeling for |
| | dysplasia correction | PX | (Spica) cast design and graded |
| | | | correction |
| | Scoliosis correction | CT, MR, | Surface contour and bone modeling |
| | | PX, XR | to evaluate angles and monitor |
| | | | correction |
| | Clubfoot correction | MR, PX | Surface contour modeling for |
| | | | graded correction (Ponseti) |
| | Congenital heart repair | MR, CT, | Blood pool and myocardial |
| | | US | modeling for operative planning and |
| | | | post-operative assessment |
| Plastic surgery | Breast reconstruction | PX, MR, | Surface contour mold for flap |
| | | CT | reconstruction |
| | Craniofacial | PX, CT, | Surgical planning and hardware |
| | reconstruction | MR, XR | fitting, cutting guide and implant |
| | | | modeling |
| Podiatric surgery | Osteotomy (e.g. | CT, XR, | Surgical planning and simulation |
| | metatarsal) | MR, PX | models |
| | Coalition correction | CT, XR, | Surgical planning and simulation |
| | | MR | models |
| Rehabilitation | Prosthetics | CT, XR, | MRI/EMG directed myoelectric |
| | | MR, PX | interface. fMRI planning and |
| | | | recovery surveillance |
| | Stroke rehabilitation | CT, PX, | MRI/EMG directed myoelectric |
| | | MR | interface. fMRI planning and |
| | | | recovery surveillance |
| | Occupational therapy | CT, XR, | Activity-oriented functional |
| | | MR | prosthetic/assist device modeling |
| Surgical oncology | Resection/tumor | CT, MR | Structural and neurovascular |
| | debulking | | modeling, simulation training |
| | Liver transplant | CT, MR | Resection/transplant/anastomosis |
| | | | planning, pre/post-embolization |
| | | | organ and tumor volumetry |
| Vascular surgery | AAA repair | CT, MR, | Surgical planning, surveillance, ex |
| | | US | vivo device sizing |
| | Endovascular stenting | CT, MR | Lumen/thrombus/stent modeling, |
| | | | endoleak assessment, device fitting |
| Urologic surgery | Bladder repair | CT, MR | Scaffold modeling, repair planning |
| | Ureter repair | CT, MR | Excretory system modeling, |
| | | | resection/re-implantation planning |
| | Renal transplant | CT, MR | Transplant/anastomosis planning |

Although example applications are shown in Table 1, these are merely illustrative examples, and local variations are expected with regard to the performing medical or surgical specialist, procedural details, and relevant diagnostic imaging. In practice, other applications of the described techniques are possible, depending on the implementation. Further, although example implementations are described with respect to particular specialties, procedures, and/or models, these are also merely illustrative examples. In practice, other implementations are all possible with respect to above described and other specialties, procedures, and/or models.

### Cardiology/Cardiothoracic Surgery:

In some cases, an adult or pediatric cardiologist may be concerned with imaging anatomy, pathology, and function of the heart and its related components. In many cases, image data from multiple modalities may be available. Information from each of these imaging modalities can be used to produce a model of the patient's heart.

For instance, ultrasound, CT, and MRI images may be available for a particular patient. Ultrasound echocardiography is often used to assess cardiac function and specific structures of the heart (e.g., the valves). CT image acquisition can be isotropic, and can be triggered prospectively or gated retrospectively to a specific portion of the cardiac cycle, in some cases diastole. MRI data sets can also be isotropic, and may also be triggered and/or gated.

CT typically provides excellent spatial resolution. However, in many cases, heart valves are often too thin to accurately visualize. Thus, 3D ultrasound of a valve can be used to supplement this weakness of CT data. Similarly, delayed myocardial enhancement on contrast enhanced MRI can relate to various pathologies including myocardial infarction, which might not be apparent in the CT data. Thus, geometries of interest related to specific anatomy and pathology can be segmented from multiple modalities, and these data can be combined and represented in a single object model or 3D printed object.

Further, geometries of interest that can be segmented relatively easily from cardiac imaging include blood pool (heart chambers, coronary arteries), myocardium, epicardial fat, heart valves, and other well-visualized anatomy and pathology. Vascular anatomy is represented in high spatial resolution by contrast-enhanced image data, especially if acquired in angiographic or other appropriate phase. In many cases, the attenuation of the contrast bolus is reliably high and allows for relatively straightforward segmentation via techniques including global thresholding, threshold painting, center-line tracing, edge tracing. Further, the contiguity of vascular trees is often well suited for region seeding and region growing segmentation techniques.

In some cases, successful preoperative planning depends on the accurate depiction of patient anatomy in a tangible or otherwise manipulable form. In these contexts, a solid or hollow 3D printed model can be used as a part of the planning process. For example, this may be a hollow model, split in pieces, that displays an inner contour representing heart chambers or vessels. In some cases, this may be a transparent or translucent model. In some cases, this may also entail a computerized model (e.g., a 3D object model file); this model can be electronically stored and retrieved, and fully manipulate by the user to study the patient's anatomy. In some cases, a model can be used for mold making and casting. When desired, a physical model may be biomimetic, and include materials and/or properties that mimic those of the patient's body.

In some cases, preoperative planning is performed in the context of medical device fitting for a patient. In this case, simulated deployment of a physical device may be performed on a model, as it would in vivo. In some implementations, to facilitate virtual deployment of a device, the patient's heart object model can be converted into a finite element mesh, and subject to finite element analysis (FEA) to simulate a dynamic interaction with the virtually deployed device.

In some cases, preoperative planning can be performed in the context of planned alterations of hemodynamics for a therapeutic goal. This may include, for example, surgical creation of a baffle or conduit to direct flow, closure of an open aperture to prevent flow, creation of a bypass route, creation of a shunt, placement of a device such a valve or filter or stent or ventricular assist device. Leveraging the flexibility of object modeling in CAD, a patient object model may be subject to the virtual procedure in the form of a simulated postoperative object model. Subsequently, a finite element mesh computerized fluid dynamics (CFD) may be simulated, thereby illustrating pre-operative and post-operative fluid dynamics. Further, in some cases, mesh simplification (e.g., mesh decimation) may precede this step to facilitate the complex computational task.

Neurology/Neurosurgery:

In some cases, neurologists or neurosurgeons may be concerned with imaging anatomy, pathology, and function of the brain, spine, nervous system, and their related components. Highly specified protocols are available to visualize complex patient anatomy and pathology. For example, in some implementations, the brainstem and cranial nerves or inner ear may be visible on a thin 2D oblique planar acquisition T2 MRI pulse sequence, and surface contours of geometries of interest (e.g., cranial nerves or inner ear structure) can be readily extracted in CAD indirectly from a model of surrounding cerebrospinal fluid (CSF), which is more easily segmented for example with global thresholding. In some cases, vascular flow voids may be visible on a proton density MRI sequence, and geometries of interest (e.g., high flow vessels) can be readily segmented by global thresholding to a low and narrow signal intensity range, thereby removing non-contiguous geometries with respect to the geometry of interest, and cropping out extraneous geometries in CAD.

In some cases, regions of pathology may only be apparent with contrast enhancement. In these situations, enhancing regions can be readily segmented using techniques such as target seeding, threshold painting, or marching cubes.

In some cases, diffusion weighted MRI pulse sequences may not contain geometries of interest, or be of adequate resolution. In some cases, susceptibility weighted MRI pulse sequences may contain diagnostically useful artifacts which may be undesirable for segmentation. In some cases, complex skull base and sinus bony anatomy may be visible with helical CT acquisition, thin multi-planar reconstruction images in bone kernel, with post-processing enhancements such as edge enhancement, surface contour rendering, or average intensity rendering; in these cases, bones can be readily segmented using global thresholding with removal of non-contiguous or extraneous geometries.

Other specific protocols may be available to image anatomy, pathology, or function related to brain, sinuses, cranial nerves, CSF, pituitary gland, hippocampi, sella tursica, temporal bones, facial bones, spine, vessels, or any other anatomy, pathology, or function that might concern a neurologist and/or neurosurgeon.

### Orthopedic Surgery

In some cases, orthopedic surgeons may concern themselves with imaging anatomy, pathology, and function of the extremities and spine, and their related components. Skeletal anatomy is often represented in high spatial resolution by CT, which can be acquired as an isotropic data set. Attenuation characteristics of bone are well established in the medical literature, and are typically normalized to water (e.g., 0 HFU) on CT data sets. Soft tissue anatomy is often well depicted by MRI, which can also be acquired as an isotropic data set. These are general considerations, and segmentation steps can be highly dependent on the representational goals of the anatomic model.

In some cases, standard orthopedic hardware must be fit to anatomy of a specific patient, for example in shaping a fixation plate to conform to bone contours of a specific patient. A printed model can therefore serve as a template for shaping the hardware prior to the actual procedure, reducing operating time that would otherwise be spent shaping the hardware operatively.

In some cases a portion of an extremity may be planned for resection, for example excision of an intra-osseous tumor. The extent of bone involvement can be visualized with CT, and contrast-enhanced MRI can provide an indication of the necessary extent of the resection to remove the tumor with adequate surgical margins. Once these margins are determined, an object model of the bone can be made, and from this object model a prosthesis model representing a surgical cutting guide can be made to ensure the resection is performed as planned. The portion of the object model that will be excised can be isolated and subsequently used to create a second prosthesis model, which can then be used to fabricate a titanium bone implant corresponding to the surgical defect. Known or approximated mechanical properties of the implant model and object model (with surgical defect) can be used to assess the mechanical properties of the post-operative extremity using finite element analysis, providing insight into mechanical interaction of the implant and extremity following the procedure. This may be especially relevant in weight-bearing extremities, where post-operative mechanical changes related to an implanted device can lead to stress shielding and subsequent undesirable morphologic re-modeling. If outcomes such as this are suspected based on the FEA of the component meshes, this information can be integrated into iterative redesign of the planned surgical defect, informed by both the necessary surgical margins for resection as well as force distribution within the post-operative extremity.

### Rehabilitation:

In some cases, a person may suffer from deformity or the partial or total loss of a body part (e.g., a limb, joint, hand, feet, digit, tooth, or other part of his body), suffer an injury to that body part (e.g., a dislocated joint or fractured bone), or suffer functional impairment of that body part (e.g., a motor deficit following a stroke). These patients can be treated using prostheses. As an example, a person who is missing a leg can use a prosthetic leg to assist with standing, walking, swimming, or performing other tasks that might otherwise be difficult without two legs. As another example, a person who has injured an arm can use a prosthesis to provide external support for the arm, such that the arm is allowed to heal more effectively. As another example, a person suffering motor deficits after a stroke can use functional prosthesis to assist with motor function (e.g. neuromuscular training) and functional rehabilitation, and to prevent development of contractures. Rehabilitation devices such as this may also be more specifically designed for occupational therapy priorities, which can be more relevant to the patient by designing a prosthesis to meet specific functional requirements for daily living.

### Educational/Training Tools:

In some cases, computerized and/or physical models of a patient can be used as educational tools. For example, models of a patient can be provided to healthcare trainees to help them visualize the anatomy of a patient, without subjecting the patient to an invasive procedure. As another example, models of a patient can be used to simulate procedures and techniques, thereby allowing relatively inexperienced physicians to practice without fear of harming a patient. As yet another example, models of a patient exhibiting complex/variant anatomy and/or pathology can facilitate useful educational opportunities at one time accessible only through posthumous autopsy. In a similar manner, forensic models can be made to demonstrate specific pathologies relevant to a traumatic event or other patient outcome, facilitating communication with a non-medical audience—for example a courtroom jury.

### Example Systems

A variety of systems can be used to implement one or more of the above described techniques. For example, FIG. 6A shows a system 600 that includes a computer 602, a camera 604, and a 3D printer 506. In this example, each of the components of the system 600 are positioned in close proximity of each other and are local to the patient. The patient (or his caretaker) can use the camera 604 to photograph the body part of interest, and transmit the photographs of the computer 602. The computing device 604 uses the photographs to generate an object model and/or prosthesis model, and generate a set of instructions for the 3D printer 606 (e.g., as described above in reference to processes 100, 200, and 300). As an example, the computing device 604 can perform photogrammetry techniques on the photographs and generate object models. As another example, the computing device 604 can store data corresponding other patients, and can compare information regarding the present patient against information regarding the other patients in order to identify supplemental data. Once a set of instructions has been generated, the set of instructions is transmitted from the computer 602 to the 3D printer 606. The 3D printer 606 executes the set of instructions, and produces a prosthesis.

As described above, each of the components of example system 600 are in close proximity to each other. For example, each of the components can be in the same room or same general location, such that a person can readily access each of the components of system 600. Each of the components of system 600 can be interconnected, for example, using a wired connection (e.g., through an Ethernet cable, serial cable, direct connection, and so forth) or a wireless connection (e.g., though WiFi, Bluetooth, NFC, and so forth).

In the above example, the computer 602 locally stores data corresponding to other patients, and uses this information to identify supplemental data. However, this need not be the case. For example, as shown in FIG. 5B, another example system 620 includes a server computer 608. Server computer 608 can be local to or remote from the other components of system 620. For example, the server computer 608 can be in the same room as the other components of system 620, in a different room, in a different building, or in a different city or country entirely, depending on the implementation. Server computer 608 is interconnected with the computer 602 through a network 510. The network 510 can be, for example, a local area network (e.g., an Ethernet network, local WiFi network, and so forth) a wide area network (e.g., the Internet), a cellular network, or other type of network that can transmit data between the server computer 608 and the computer 602. Server computer 608 can perform one or more of the tasks described above. For example, in some implementations, the server computer 608 can store data corresponding to other patients; computer 602 can communicate with server computer 608 to retrieve data corresponding to other patients, and to identify supplemental data for use in generating an object model. As another example, in some implementations, the server computer 608 can receive information from computer 602 (e.g., demographic and imaging information regarding the present patient), and use this information to identify supplemental information, generate an object model, and/or generate a set of instructions for the 3D printer 606. The supplemental information, object model, and/or set of instructions is then transmitted back to the computer 602. In this manner, one or more of the tasks that might otherwise be performed by the computer 602 can instead be performed by the server 608. This can be beneficial, for example, if the computer 602 is relatively less powerful than the server computer 608. In some implementations, it may also be beneficial to use the server 608 as a common repository for data pertaining to multiple different patients, such that a centralized database of demographic information, imaging information, and other patient information can be used to identify potential supplemental data. As an example, one or more computers 602 can access server computer 608 in order to obtain supplemental data.

Another example system 640 is shown in FIG. 5C. System 640 is similar to system 620, and includes a server computer 608 in communication with computer 602 through a network 510. System 640 also includes a portable electronic device 612 in communication with the computer 602 and server computer 608 (either through the network 510 or a separate network). As with server 608, the electronic device 612 can be local or remote to one or more other components of system 640. In some implementations, the electronic device 612 can be used to control aspects of the computer 602, the server 608, and/or the 3D printer 606. For instance, a user can input commands into the electronic device 612 in order to transmit images to the computer 602 or server computer 608, to transmit sets of printing instructions to the computer 602 or 3D printer 606, to transmit a command to begin printing to computer 602 or 3D printer 606, or any other command in order to perform one or more of the tasks described above. In some implementations, the electronic device 612 also includes a camera (e.g., a built in camera module or a connected discrete camera). In these implementations, a user can use the camera of electronic device 612 in order to capture images of a patient. These images can then be transmitted to the computer 602 and/or the server computer 608 for further processing. Thus, a patient need only be local to the electronic device 612, while the other components of system 640 can be located elsewhere.

Several example system configurations are shown above, and each may be particularly suitable under certain circumstances. As an example, the system 600 shown in FIG. 5A might be suitable if the patient has local access to a relatively powerful computer 602, a camera 604, and a 3D printer 606. Further, the system 600 might be suitable if the patient is trained to operate the system 600, or has local access to a capable operator. Further, the system 600 might be suitable if the patient has access to sufficient electricity and other utility infrastructure to operate the system 600.

As another example, the system 620 shown in FIG. 5B might be appropriate if the patient is located in an area with access to a relatively weak computer 602, but otherwise has access to a 3D printer 606, a camera 604, and a data network 510 in which to communicate with server 608. The system 620 might also be appropriate if several different computers 602 are being used in multiple different locations; in this case, a server 608 might be beneficial in order to consolidate information regarding a large number of patients into a centralized database.

As another example, the system 640 shown in FIG. 5C might be appropriate if the patient is located in an area with little or no access to computers, 3D printers, or reliable electricity and/or network infrastructure. In this implementation, a patient need only be local to the electronic device 612 (which may be portable and battery powered), and a prosthesis can be produced remotely based on photographs or other information captured by the electronic device 612.

Although several system configurations are shown above, these are only examples to illustrate how various components of a system can be positioned either local to each other or remote to each other, depending on the application. Other system configurations are possible.

Further, although examples of how the process of producing a prosthetic device can be distributed between various components of a system as described above, these also are only examples. In practice, each aspect of producing a prosthetic device can be performed by one or more components of the system, either independently or in conjunction with other local or remote components. For example, aspects of the process of producing a prosthetic device can be performed, in part, using cloud computing-based resources, remote desktop-based resources, or server-based resources, in which processing capability is provided at location(s) remote to the user and/or patient.

Some implementations of subject matter and operations described in this specification can be implemented in digital electronic circuitry, or in computer software, firmware, or hardware, including the structures disclosed in this specification and their structural equivalents, or in combinations of one or more of them. For example, in some implementations, computer 602, server computer 606, and/or electronic device 612 can be implemented using digital electronic circuitry, or in computer software, firmware, or hardware, or in combinations of one or more of them. In another example, processes 100, 200, 300, and 500 can be implemented using digital electronic circuitry, or in computer software, firmware, or hardware, or in combinations of one or more of them.

Some implementations described in this specification can be implemented as one or more groups or modules of digital electronic circuitry, computer software, firmware, or hardware, or in combinations of one or more of them. Although different modules can be used, each module need not be distinct, and multiple modules can be implemented on the same digital electronic circuitry, computer software, firmware, or hardware, or combination thereof.

Some implementations described in this specification can be implemented as one or more computer programs, i.e., one or more modules of computer program instructions, encoded on computer storage medium for execution by, or to control the operation of, data processing apparatus. A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Some of the processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and processors of any kind of digital computer. Generally, a processor will receive instructions and data from a read only memory or a random access memory or both. A computer includes a processor for performing actions in accordance with instructions and one or more memory devices for storing instructions and data. A computer may also include, or be operatively coupled to receive data from or transfer data to, or both, one or more mass storage devices for storing data, e.g., magnetic, magneto optical disks, or optical disks. However, a computer need not have such devices. Devices suitable for storing computer program instructions and data include all forms of non-volatile memory, media and memory devices, including by way of example semiconductor memory devices (e.g., EPROM, EEPROM, flash memory devices, and others), magnetic disks (e.g., internal hard disks, removable disks, and others), magneto optical disks, and CD ROM and DVD-ROM disks. The processor and the memory can be supplemented by, or incorporated in, special purpose logic circuitry.

To provide for interaction with a user, operations can be implemented on a computer having a display device (e.g., a monitor, or another type of display device) for displaying information to the user and a keyboard and a pointing device (e.g., a mouse, a trackball, a tablet, a touch sensitive screen, or another type of pointing device) by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A computer system may include a single computing device, or multiple computers that operate in proximity or generally remote from each other and typically interact through a communication network. Examples of communication networks include a local area network (“LAN”) and a wide area network (“WAN”), an inter-network (e.g., the Internet), a network comprising a satellite link, and peer-to-peer networks (e.g., ad hoc peer-to-peer networks). A relationship of client and server may arise by virtue of computer programs running on the respective computers and having a client-server relationship to each other.

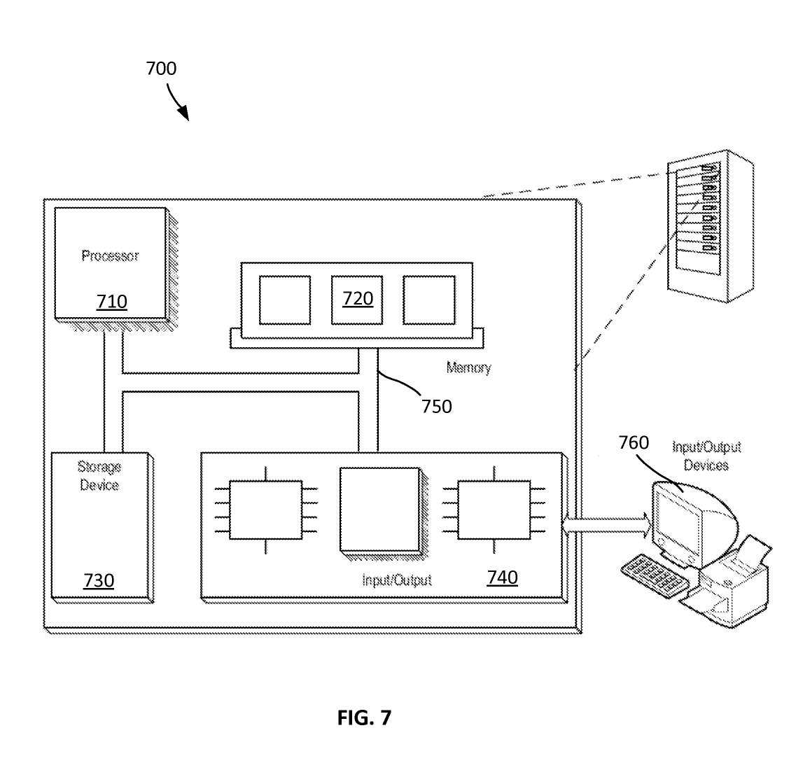

FIG. 7 shows an example computer system 700. The system 700 includes a processor 710, a memory 720, a storage device 730, and an input/output device 740. Each of the components 710, 720, 730, and 740 can be interconnected, for example, using a system bus 750. The processor 710 is capable of processing instructions for execution within the system 700. In some implementations, the processor 710 is a single-threaded processor, a multi-threaded processor, or another type of processor. The processor 710 is capable of processing instructions stored in the memory 720 or on the storage device 730. The memory 720 and the storage device 730 can store information within the system 600.

The input/output device 740 provides input/output operations for the system 700. In some implementations, the input/output device 740 can include one or more of a network interface devices, e.g., an Ethernet card, a serial communication device, e.g., an RS-232 port, and/or a wireless interface device, e.g., an 802.11 card, a 3G wireless modem, a 4G wireless modem, etc. In some implementations, the input/output device can include driver devices configured to receive input data and send output data to other input/output devices, e.g., keyboard, printer and display devices 760. In some implementations, mobile computing devices, mobile communication devices, and other devices can be used.

While this specification contains many details, these should not be construed as limitations on the scope of what may be claimed, but rather as descriptions of features specific to particular examples. Certain features that are described in this specification in the context of separate implementations can also be combined. Conversely, various features that are described in the context of a single implementation can also be implemented in multiple embodiments separately or in any suitable subcombination.

A number of implementations have been described. Nevertheless, it will be understood that various modifications may be made without departing from the spirit and scope of the invention. Accordingly, other implementations are within the scope of the following claims.

1. A method for producing a prosthetic device for a patient, the method comprising:
obtaining imaging data corresponding to a body part of a patient;
generating an object model corresponding to the body part based on the imaging data;
generating a prosthesis model based on the object model;
generating a set of instructions based on the prosthesis model; and
executing the set of instructions using a three-dimensional printer, wherein the set of instructions, when executed by the three-dimensional printer, cause the three-dimensional printer to produce the prosthetic device for the patient.

2. The method of claim 1, further comprising:
identifying supplemental data based on the received imaging data and/or the object model, wherein supplemental data comprises supplemental imaging data corresponding to body parts of one or more other patients; and
updating the object model based on the imaging data and the supplemental data.

3. The method of claim 1, further comprising:
identifying supplemental data based on the received imaging data, wherein supplemental data comprises supplemental imaging data corresponding to body parts of one or more other patients; and
wherein generating an object model is further based on supplemental data

4. The method of claim 2, wherein the imaging data and the supplemental imaging data are each acquired using the same imaging modality.

5. The method of claim 2, wherein the imaging data and the supplemental imaging data are each acquired using a different imaging modality.

6. The method of claim 4, wherein the imaging modality is photography, computed tomography, magnetic resonance imaging, or X-ray.

7-9. (canceled)

10. The method of claim 2, wherein the imaging data and the supplemental imaging data correspond to similar body parts.

11. The method of claim 1, wherein the object model comprises surface information, mesh information, or information regarding a plurality of components.

12-14. (canceled)

15. The method of claim 1, wherein generating the object model comprises determining the object model using photogrammetry.

16. The method of claim 1, wherein generating the object model comprises segmenting the imaging data into one or more portions, each portion corresponding to a different bone or soft tissue structure in the body part.

17. (canceled)

18. The method of claim 1, wherein generating the object model comprises modeling a surface of the body part or estimating a volume of the body part.

19. (canceled)

20. The method of claim 2, wherein identifying supplemental data comprises determining a similarity between the supplemental imaging data and the imaging data.

21-24. (canceled)

25. The method of claim 2, wherein identifying supplemental data comprises determining a similarity between demographic data corresponding the patient and demographic data corresponding to one or more other patients.

26. The method of claim 1, wherein the set of instructions, when executed by the three-dimensional printer, cause the three-dimensional printer to produce the prosthetic device for the patient through an additive manufacturing process.

27. The method of claim 1, wherein generating the object model comprises:
transmitting the imaging data to a remote processing device; and
receiving the object model from the remote processing device, wherein the object model is generated by the remote processing device based on the imaging data.

28. The method of claim 1, wherein generating a prosthesis model based on the object model comprises:
transmitting the object model to a remote processing device; and
receiving the prosthesis model from the remote processing device, wherein the prosthesis model is generated by the remote processing device based on the object model.

29. The method of claim 1, wherein generating the set of instructions based on the object model comprises:
transmitting the prosthesis model to a remote processing device; and
receiving the set of instructions from the remote processing device, wherein the set of instructions is generated by the remote processing device based on the prosthesis model.

30. The method of claim 1, wherein generating the object model, generating the prosthesis model based on the object model, and generating the set of instructions based on the prosthesis model comprises generating the object model, the prosthesis model, and the set of instructions through the use of a remote processing device.

31. A system for producing a prosthetic device for a patient, the system comprising:
one or more data processing apparatuses configured to:
obtain imaging data corresponding to a body part of a patient;
generate an object model corresponding to the body part based on the imaging data;
generate a prosthesis model based on the object model;
generate a set of instructions based on the prosthesis model; and
execute the set of instructions using a three-dimensional printer, wherein the set of instructions, when executed by the three-dimensional printer, cause the three-dimensional printer to produce the prosthetic device for the patient.

32-60. (canceled)

61. A method for producing an object model of a patient, the method comprising:
obtaining a first set of imaging data corresponding to a body part of a patient, wherein the first set of imaging data was acquired using a first imaging modality;
obtaining a second set of imaging data corresponding to the body part of the patient, wherein the second set of imaging data was acquired using a second imaging modality different than the first imaging modality; and
generating an object model corresponding to the body part based on the first set of imaging data and the second set of imaging data.

62-82. (canceled)
